(19) United States
(12) Patent Application Publication
(10) Pub. No.: US 2025/0336523 A1
(43) Pub. Date: Oct. 30, 2025

(54) APPARATUS AND METHODS FOR GENERATING DIAGNOSTIC HYPOTHESES BASED ON BIOMEDICAL SIGNAL DATA

(71) Applicant: Anumana, Inc., Cambridge, MA (US)

(72) Inventors: Akash Anand, Bhagalpur (IN); Avinash Aman, Domlur (IN); Nikhil Sachdeva, Murugeshpallya (IN); Deep Hitesh Wankawala, Jeevan Bima Nagar (IN)

(73) Assignee: Anumana, Inc., Cambridge, MA (US)

(21) Appl. No.: 18/648,059

(22) Filed: Apr. 26, 2024

(51) Int. Cl. G16H 50/20; A61B 5/00; A61B 5/346; G16H 10/60

(52) U.S. Cl. CPC G16H 50/20; A61B 5/346; A61B 5/7267; G16H 10/60

(57) ABSTRACT

An apparatus for generating diagnostic hypotheses based on electrocardiogram (ECG) data, comprising a processor and a memory containing instructions configuring the processor to generate, using a generative model trained on a corpus, a set of diagnostic hypotheses, wherein generating the set of diagnostic hypotheses includes creating labels, each represents a diagnostic feature associated with diagnostic hypotheses, receive a biomedical signal, identify a biomedical feature as a function of the biomedical signal, select a diagnostic hypothesis from the set of diagnostic hypotheses by matching the biomedical feature against the diagnostic feature, query, as a function of at least a matched label, a medical repository to validate the diagnostic hypothesis, wherein the medical repository includes patients' electronic health records (EHRs), and output the diagnostic hypothesis upon a positive validation of the diagnostic hypothesis.

### FIELD OF THE INVENTION

The present invention generally relates to the field of machine learning in medical diagnostics. In particular, the present invention is directed to an apparatus and methods for generating diagnostic hypotheses based on biomedical signal data.

### BACKGROUND

Diagnosis of medical conditions has relied heavily on the manual interpretation of biomedical signals by trained healthcare professionals. Electrocardiograms (ECGs), for example, are often time used to assess the electrical activity of the heart and detect various cardiac conditions. However, the interpretation of ECGs can be challenging due to the subtlety of certain cardiac abnormalities and the potential for human error.

### SUMMARY OF THE DISCLOSURE

In an aspect, an apparatus for generating diagnostic hypotheses based on electrocardiogram (ECG) data is described. The apparatus includes at least a processor and a memory communicatively connected to the at least a processor, wherein the memory contains instructions configuring the at least a processor to generate, using a generative model trained on a corpus, a set of diagnostic hypotheses, wherein generating the set of diagnostic hypotheses includes creating a plurality of labels, wherein each label of the plurality label represents at least one diagnostic feature associated with one or more diagnostic hypotheses within the set of diagnostic hypotheses. The processor is configured to receive a biomedical signal pertaining to a patient, identify at least one biomedical feature as a function of the biomedical signal, select at least one diagnostic hypothesis from the set of diagnostic hypotheses for the patient by matching the at least one biomedical feature against the at least one diagnostic feature, query, as a function of at least a matched label, a medical repository in communication with the processor, to validate the at least one diagnostic hypothesis, wherein the medical repository includes a plurality of electronic health records (EHRs) associated with a plurality of patients. The processor is further configured to output the at least one diagnostic hypothesis upon a positive validation of the at least one diagnostic hypothesis.

In another aspect, a method for generating diagnostic hypotheses based on electrocardiogram (ECG) data is described. The method includes generating, by at least a processor, a set of diagnostic hypotheses using a generative model trained on a corpus, wherein generating the set of diagnostic hypotheses includes creating a plurality of labels, wherein each label of the plurality label represents at least one diagnostic feature associated with one or more diagnostic hypotheses within the set of diagnostic hypotheses. The method includes receiving, by the at least a processor, a biomedical signal pertaining to a patient, identifying, by the at least a processor, at least one biomedical feature as a function of the biomedical signal, selecting, by the at least a processor, at least one diagnostic hypothesis from the set of diagnostic hypotheses for the patient by matching the at least one biomedical feature against the at least one diagnostic feature, querying, by the at least a processor, a medical repository in communication with the processor as a function of at least a matched label to validate the at least one diagnostic hypothesis, wherein the medical repository includes a plurality of electronic health records (EHRs) associated with a plurality of patients. The method further includes outputting, by the at least a processor, the at least one diagnostic hypothesis upon a positive validation of the at least one diagnostic hypothesis.

These and other aspects and features of non-limiting embodiments of the present invention will become apparent to those skilled in the art upon review of the following description of specific non-limiting embodiments of the invention in conjunction with the accompanying drawings.

### BRIEF DESCRIPTION OF THE DRAWINGS

For the purpose of illustrating the invention, the drawings show aspects of one or more embodiments of the invention. However, it should be understood that the present invention is not limited to the precise arrangements and instrumentalities shown in the drawings, wherein:

FIG. 1 illustrates a block diagram of an exemplary embodiment of an apparatus for generating diagnostic hypotheses based on electrocardiogram (ECG) data.

FIG. 2 illustrates an exemplary embodiment of a user interface;

FIG. 3 illustrates an exemplary embodiment of a user interface;

FIG. 4 illustrates an exemplary embodiment of a user interface;

FIG. 5 illustrates an exemplary embodiment of a user interface;

FIG. 6 illustrates an exemplary embodiment of a user interface;

FIG. 7 illustrates a block diagram of an exemplary embodiment of a chatbot;

FIG. 8 illustrates a block diagram of exemplary embodiment of a machine learning module;

FIG. 9 illustrates a diagram of an exemplary nodal network;

FIG. 10 illustrates a block diagram of an exemplary node;

FIG. 11 illustrates a flow diagram of an exemplary embodiment of a method for generating diagnostic hypotheses based on electrocardiogram (ECG) data; and

FIG. 12 is a block diagram of a computing system that can be used to implement any one or more of the methodologies disclosed herein and any one or more portions thereof.

The drawings are not necessarily to scale and may be illustrated by phantom lines, diagrammatic representations, and fragmentary views. In certain instances, details that are not necessary for an understanding of the embodiments or that render other details difficult to perceive may have been omitted.

### DETAILED DESCRIPTION

At a high level, aspects of the present disclosure are directed to an apparatus and methods for generating diagnostic hypotheses based on electrocardiogram (ECG) data. In an embodiment, apparatus is configured to analyze biomedical signal, extract biomedical features, and match extracted features against a set of diagnostic hypotheses derived from a corpus of medical literature.

Aspects of the present disclosure can be used to streamline the diagnostic process by automating the analysis of biomedical signal such as ECG data and reducing reliance on manual interpretation. Aspects of the present disclosure can also be used to enhance the accuracy of diagnoses by employing a generative model such as a large language model (LLM) which is trained on corpus and validated against real-world patient outcomes. This is so, at least in part, because the disclosed apparatus and method utilize the generative model to interpret biomedical signals and validate diagnostic hypotheses against up-to-date medical knowledge and patient records from medical repositories.

Aspects of the present disclosure allow for a personalized approach to patient care by leveraging de-identified data from extensive patient cohorts to identify similar cases and contextualize individual patient diagnoses within broader population health data. Exemplary embodiments illustrating aspects of the present disclosure are described below in the context of several specific examples.

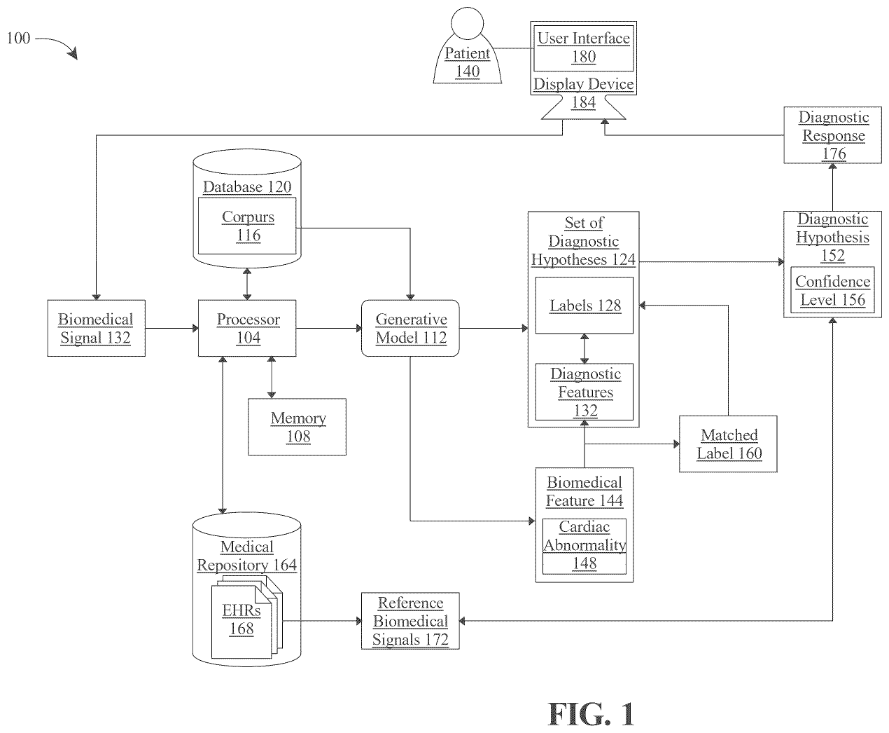

Referring now to FIG. 1, an exemplary embodiment of an apparatus 100 for generating diagnostic hypotheses based on electrocardiogram (ECG) data is illustrated. Apparatus 100 includes a computing device. Computing device includes a processor 104 communicatively connected to a memory 108. As used in this disclosure, “communicatively connected” means connected by way of a connection, attachment, or linkage between two or more relata which allows for reception and/or transmittance of information therebetween. For example, and without limitation, this connection may be wired or wireless, direct, or indirect, and between two or more components, circuits, devices, systems, and the like, which allows for reception and/or transmittance of data and/or signal(s) therebetween. Data and/or signals therebetween may include, without limitation, electrical, electromagnetic, magnetic, video, audio, radio, and microwave data and/or signals, combinations thereof, and the like, among others. A communicative connection may be achieved, for example and without limitation, through wired or wireless electronic, digital, or analog, communication, either directly or by way of one or more intervening devices or components. Further, communicative connection may include electrically coupling or connecting at least an output of one device, component, or circuit to at least an input of another device, component, or circuit. For example, and without limitation, via a bus or other facility for intercommunication between elements of a computing device. Communicative connecting may also include indirect connections via, for example and without limitation, wireless connection, radio communication, low power wide area network, optical communication, magnetic, capacitive, or optical coupling, and the like. In some instances, the terminology “communicatively coupled” may be used in place of communicatively connected in this disclosure.

With continued reference to FIG. 1, processor 104 may include any computing device as described in this disclosure, including without limitation a microcontroller, microprocessor, digital signal processor (DSP) and/or system on a chip (SoC) as described in this disclosure. Processor 104 may include, be included in, and/or communicate with a mobile device such as a mobile telephone or smartphone. Processor 104 may include a single computing device operating independently, or may include two or more computing device operating in concert, in parallel, sequentially or the like; two or more computing devices may be included together in a single computing device or in two or more computing devices. Processor 104 may interface or communicate with one or more additional devices as described below in further detail via a network interface device. Network interface device may be utilized for connecting processor 104 to one or more of a variety of networks, and one or more devices. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network may employ a wired and/or a wireless mode of communication. In general, any network topology may be used. Information (e.g., data, software etc.) may be communicated to and/or from a computer and/or a computing device. Processor 104 may include but is not limited to, for example, a computing device or cluster of computing devices in a first location and a second computing device or cluster of computing devices in a second location. Processor 104 may include one or more computing devices dedicated to data storage, security, distribution of traffic for load balancing, and the like. Processor 104 may distribute one or more computing tasks as described below across a plurality of computing devices of computing device, which may operate in parallel, in series, redundantly, or in any other manner used for distribution of tasks or memory between computing devices. Processor 104 may be implemented, as a non-limiting example, using a “shared nothing” architecture.

With continued reference to FIG. 1, processor 104 may be designed and/or configured to perform any method, method step, or sequence of method steps in any embodiment described in this disclosure, in any order and with any degree of repetition. For instance, processor 104 may be configured to perform a single step or sequence repeatedly until a desired or commanded outcome is achieved; repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks. Processor 104 may perform any step or sequence of steps as described in this disclosure in parallel, such as simultaneously and/or substantially simultaneously performing a step two or more times using two or more parallel threads, processor cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

With continued reference to FIG. 1, apparatus 100 and/or processor 104 may perform determinations, classification, and/or analysis steps, methods, processes, or the like as described in this disclosure using machine learning processes. A “machine learning process,” as used in this disclosure, is a process that automatedly uses a body of data known as “training data” and/or a “training set” (described further below) to generate an algorithm that will be performed by processor 104 or module to produce outputs given data provided as inputs; this is in contrast to a non-machine learning software program where the commands to be executed are determined in advance by a user and written in a programming language. Machine-learning process may utilize supervised, unsupervised, lazy-learning processes and/or neural networks, described further below. In one embodiment, apparatus 100 and/or processor 104 is configured to implement a generative model 112. As used in this disclosure, a “generative model” is a type of machine learning process designed to create, establish, or otherwise generate new data samples that resemble the training data. Exemplary generative models may include, without limitation, generative adversarial networks (GANs), variational autoencoders (VAEs), large language model (LLM), and the like. In some cases, training examples may encompass a diverse range of data modality e.g., text, images, video, audio, sequences, signals, and/or the like. Apparatus 100 and/or processor 104 may be configured to implement a plurality of generative models (one or more generative models for each data modality), for example, and without limitation, GANs for image data and LLMs for textural or complex sequence data. In some cases, different generative model may be selected and implemented based on specific requirements of the data type being processed and analyzed. As a person skilled in the art, upon reviewing the entirety of this disclosure, will be aware of various generative models suitable for different application across various domains.

With continued reference to FIG. 1, as a non-limiting example, processor may be configured to implement a large language model (LLM). A “large language model,” as used herein, is a deep learning data structure that can recognize, summarize, translate, predict and/or generate text and/or other content based on knowledge gained from massive datasets. LLM may be trained on large sets of data. In one embodiment, generative model 112 is trained on a corpus 116. As used in this disclosure, a “corpus” is a large set of data. Corpus data may include text, images, videos, audio, or the like. Corpus data may be structured, semi-structure, and/or unstructured. In some cases, corpus 116 may include a collection of sufficiently diverse and comprehensive texts, covering desired breadth and depth of knowledge to one or more domains (e.g., medicine including cardiology, pharmacology, epidemiology, and the like), that is used to train LLM, allowing LLM to understand, interpret, and/or generate language-based outputs that are relevant to the model's intended applications as described herein. In some cases, corpus 116 may include a set of medical literatures encompassing research findings, clinical studies, reviews, case reports, scholarly articles, and any other written material related to the field of medicine and healthcare. As a non-limiting example, corpus 116 may include a collection of peer-reviewed medical research papers, reviewed, articles from reputable journals, official clinical guidelines, treatment protocols, best practice documents from recognized medical associations and/or organizations, medical textbooks, reference materials covering explanation of medical conditions, treatments, health maintenance strategies, online medical forums from online medical communities including discussions and Q&A sessions, among others. In some cases, corpus 116 may include information from one or more public or private databases. As a non-limiting example, corpus may include a PubMed database or any other repository of knowledge within medical community.

With continued reference to FIG. 1, in one or more embodiments, processor 104 may be configured to access one or more databases 120. As described herein, a “database” is a collection of data that can be accessed, managed, and updated. In one or more embodiments, database 120 may include one or more systematically organized collections of medical literatures and/or patient records as described herein, interfacing with processor 104 and one or more other data storage mechanisms, which may be efficiently retrieved, updated, and/or manipulated. As a non-limiting example, database 120 may include a relational database having one or more structured formats that organize set of medical literatures and/or patient records into one or more tables with plurality of rows and columns. Apparatus 100 may implement one or more aspect of a database management system (DBMS), for example and without limitation, functions such as data element insertion, querying, update, delete, and administration may be implemented and performed, by processor 104, on database 120. In some embodiments, database 120 may include flexible schemas e.g., key-value stores. In some cases, processor 104 may access one or more data warehouses or data lakes or repositories that report data analytics or hold a large amount of raw data in its native format until needed. Additionally, or alternatively, database 120 may include one or more datasets or “corpora,” collections of values, written texts, recorded speech, or the like. As a non-limiting example, database 112 may be implemented, without limitation, as a relational database, a key-value retrieval database such as a NOSQL database, or any other format or structure for use as a database that a person skilled in the art would recognize as suitable upon review of the entirety of this disclosure. Database may alternatively or additionally be implemented using a distributed data storage protocol and/or data structure, such as a distributed hash table or the like. Database may include a plurality of data entries and/or records such as, without limitation, set of medical literatures and/or patient records as described herein. Data entries in a database may be flagged with or linked to one or more additional elements of information, which may be reflected in data entry cells and/or in linked tables such as tables related by one or more indices in a relational database. Persons skilled in the art, upon reviewing the entirety of this disclosure, will be aware of various ways in which data entries in database 120 may store, retrieve, organize, and/or reflect data elements as used herein, as well as categories and/or populations of data consistently with this disclosure.

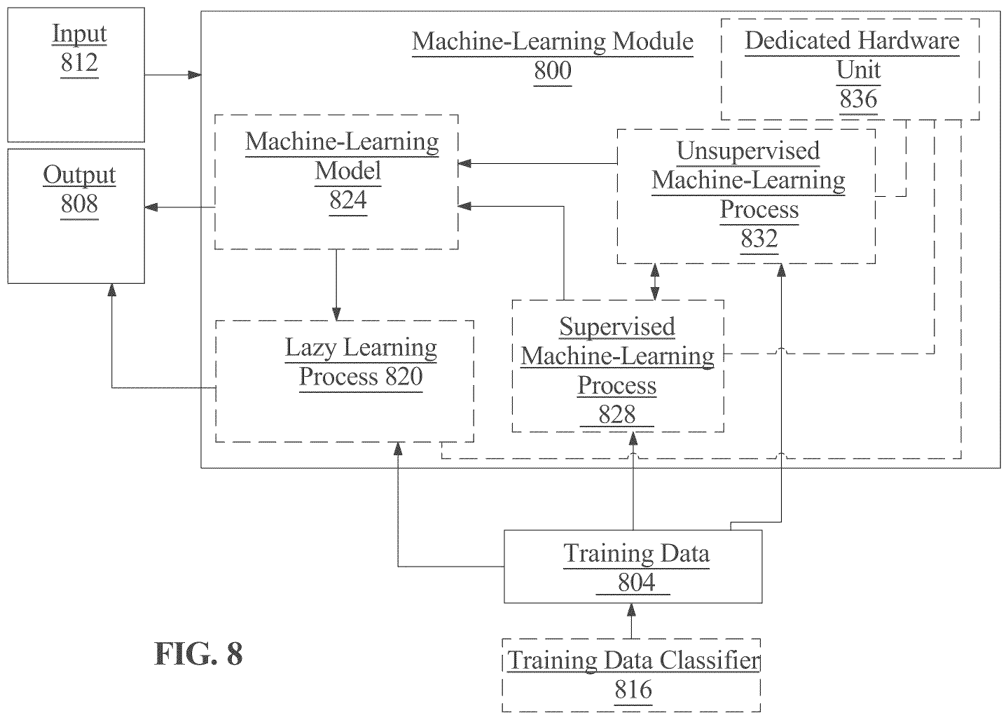

With continued reference to FIG. 1, in some embodiments, generative model 112 such as, without limitation, LLM may be generally trained. As used in this disclosure, a “generally trained” model is a model that is trained on a general training set comprising a variety of subject matters, data sets, and fields. In some embodiments, LLM may be initially generally trained. Additionally, or alternatively, generative model 112 may be specifically trained. As used in this disclosure, a “specifically trained” or “specially trained” model is a model that is trained on a specific training set, wherein the specific training set includes data including specific correlations for the model to learn. As a non-limiting example, a LLM may be generally trained on a general training set, then specifically trained on a specific training set. In an embodiment, specific training of generative model 112 may be performed using a supervised machine learning process. In some embodiments, generally training generative model 112 may be performed using an unsupervised machine learning process. Supervised and unsupervised machine learning is described in further detail below with reference to FIG. 8. As a non-limiting example, generative model 112 such as, without limitation, a LLM may be pre-training on a general set of medical literatures (i.e., a wide range of medical literatures covering the vast field of medicine) and fine-tuning on a specific set of medical literatures (i.e., texts focused on one or more specific areas), wherein the general set of medical literatures and the specific set of medical literatures are subsets of the set of medical literatures contained in corpus 116. In some cases, majority of medical literatures within the specific set may be more detailed, advanced, or specialized then medical literatures in the general set. For example, general set of medical literatures may include introductory texts and reviews that summarize basic concepts in physiology while specific set of medical literatures may include a plurality of citations to peer-reviewed research papers related to cardiology.

With continued reference to FIG. 1, in one embodiment, training generative model 112, such a LLM may include setting one or more parameters of the one or more models (weights and biases) either randomly or using a pretrained model. In some cases, generally training LLM on a large corpus of text data e.g., general set of medical literatures may provide a starting point for fine-tuning on a specific task. LLM may learn by adjusting its parameters during the training process to minimize a defined loss function, which measures the difference between predicted outputs and ground truth. Once a model has been generally trained (i.e., pre-trained model), LLM may then be specifically trained to fine-tune the pretrained model on task-specific data e.g., specific set of medical literatures to adapt it to one or more target tasks by adjusting the weights to optimize performance for the target tasks. In some cases, this may include optimizing the LLM performance by fine-tuning hyperparameters such as learning rate, batch size, and regularization. Hyperparameter tuning may help in achieving the best performance and convergence during training. In one or more embodiments, fine-tuning LLM may include fine-tuning the pretrained model using Low-Rank Adaptation (LoRA). As used in this disclosure, “Low-Rank Adaptation” is a training technique for large language models that modifies a subset of parameters in the model. Low-Rank Adaptation may be configured to make the training process more computationally efficient by avoiding a need to train an entire model from scratch. In an exemplary embodiment, a subset of parameters that are updated may include parameters that are associated with a specific task or domain.

With continued reference to FIG. 1, as a non-limiting example, fine-tuning LLM may include freezing a pre-trained weight matrix (W₀) of a layer of a pre-trained model and determining an accumulated gradient update (ΔW) of the layer during adaptation of the pre-trained weight matrix. W₀ may be a matrix with W₀∈R^{d×k}. ΔW may be a matrix with the same dimensions as W₀. When running LLM, a forward pass (h) of a layer may be determined using the formula h=W₀X+ΔWX where X is the input from a previous layer. In some embodiments, only a subset of layers of LLM may be fine-tuned thereby improving the efficiency of LLM's training. LLM trained on a broad variety of data may be fine-tuned for a specific purpose; for instance, and without limitation, a LLM trained to understand and interpret general medical literature across various disciplines may be fine-tuned to specialize in generating set of diagnostic hypotheses XXX for a particular heart condition as described in further detail below. Continuing the non-limiting example, in low rank adaptation, ΔW is replaced by low rank decomposition matrices A and B, using the formula ΔW=BA. B and A may be matrices with B∈R^{d×r}, and A∈R^{r×k}. Hyperparameter r may represent the rank of a low rank adaptation module and may be chosen such that r<min(d,k) based on factors described below. A forward pass of a layer trained using low rank adaptation may have the formula h=W₀X+BAX. A random Gaussian initialization may be used to determine initial values for A and initial values of B may be set to 0, such that ΔW=BA is 0 before training. ΔWX may be scaled by α/r during training, where α is a constant in r. In some embodiments, α may be tuned as one would tune a learning rate. In some embodiments, α may be set and not tuned further. In some embodiments, a plurality of layers of a neural network may be fine-tuned using low rank adaptation. Fine-tuning a pre-trained neural network using low-rank adaptation may reduce memory and/or processing power requirements of fine-tuning the neural network, as B and A have fewer trainable parameters than ΔW would have in a non-low rank adaptation approach. In some embodiments, such difference may lead to substantial improvements where ΔW has large dimensions. The value of hyperparameter r may influence the degree to which low rank adaptation reduces memory and/or processing power requirements. In some embodiments, setting r too low may result in information loss. In some embodiments, setting r too high may result in increased memory and processing power usage for fine-tuning the neural network relative to a lower r. In some embodiments, r may be a number of linearly independent rows or columns of ΔW.

With continued reference to FIG. 1, in some cases, generative model 112 such as a LLM may include one or more architectures based on capability requirements of apparatus 100. In some cases, exemplary architectures may include, without limitation, GPT (Generative Pretrained Transformer), BERT (Bidirectional Encoder Representations from Transformers), T5 (Text-To-Text Transfer Transformer), and the like. Architecture choice may depend on a needed capability such generative, contextual, or other specific capabilities. As a non-limiting example, LLM may include and/or be produced using Generative Pretrained Transformer (GPT), GPT-2, GPT-3, GPT-4, and the like. GPT, GPT-2, GPT-3, GPT-3.5, and GPT-4 are products of Open AI Inc., of San Francisco, CA. LLM may include a text prediction based algorithm configured to receive an article and apply a probability distribution to the words already typed in a sentence to work out the most likely word to come next in augmented articles. For example, if some words that have already been typed are “the patient exhibits symptoms of chest pain and” then it may be highly likely that terms “shortness of breath” will come next.

With continued reference to FIG. 1, in some cases, generative model 112 such as a LLM may include a transformer architecture. In one or more embodiments, LLM may include an encoder component and a decoder component. In some embodiments, encoder component of LLM may include transformer architecture. A “transformer architecture,” for the purposes of this disclosure, is a neural network architecture that uses self-attention and positional encoding. Transformer architecture may be designed to process sequential input data, such as natural language, with applications towards tasks such as translation and text summarization. Transformer architecture may process the entire input all at once. In some cases, transformer may not process input sequentially, instead, it may be configured to analyze the entire input simultaneously to recognize the sequence order of input elements since the model itself does not inherently understand order in the way a recurrent neural network (RNN) does. “Positional encoding,” for the purposes of this disclosure, refers to a data processing technique that encodes the location or position of an entity in a sequence without altering the original semantic representation of sequence elements. For example, sequence element may include a word or a phrase in a sentence. In some embodiments, each position in the sequence may be assigned a unique representation. In some embodiments, positional encoding may include mapping each position in the sequence to a position vector. In some embodiments, trigonometric functions, such as sine and cosine, may be used to determine the values in the position vector. In some embodiments, position vectors for a plurality of positions in a sequence may be assembled into a position matrix, wherein each row of position matrix may represent a position in the sequence.

With continued reference to FIG. 1, in some cases, transformer architecture may include an attention mechanism. An “attention mechanism,” as used herein, is a part of a neural architecture that enables a system to dynamically quantify the relevant features of the input data. In the case of natural language processing, input data may be a sequence of textual elements. It may be applied directly to the raw input or to its higher-level representation. Attention mechanism may represent an improvement over a limitation of an encoder-decoder model. An encoder-decider model encodes an input sequence to one fixed length vector from which the output is decoded at each time step. This issue may be seen as a problem when decoding long sequences because it may make it difficult for the neural network to cope with long sentences, such as those that are longer than the sentences in the training corpus. Applying an attention mechanism, generative model 112 such as a LLM may predict the next word by searching for a set of positions in a source sentence where the most relevant information is concentrated. LLM may then predict the next word based on context vectors associated with these source positions and all the previously generated target words, such as textual data of a dictionary correlated to a prompt in a training data set. A “context vector,” as used herein, are fixed-length vector representations useful for document retrieval and word sense disambiguation.

Still referring to FIG. 1, attention mechanism may include, without limitation, generalized attention self-attention, multi-head attention, additive attention, global attention, and the like. In generalized attention, when a sequence of words or an image is fed to generative model 112, it may verify each element of the input sequence and compare it against the output sequence. Each iteration may involve the mechanism's encoder capturing the input sequence and comparing it with each element of the decoder's sequence. From the comparison scores, the mechanism may then select the words or parts of the image that it needs to pay attention to. In self-attention, generative model 112 may pick up particular parts at different positions in the input sequence and over time compute an initial composition of the output sequence. In multi-head attention, generative model 112 may include a transformer model of an attention mechanism. Attention mechanisms, as described above, may provide context for any position in the input sequence. For example, and without limitation, if the input data is a natural language sentence, the transformer does not have to process one word at a time. In multi-head attention, computations by generative model 112 may be repeated over several iterations, each computation may form parallel layers known as attention heads. Each separate head may independently pass the input sequence and corresponding output sequence element through a separate head. A final attention score may be produced by combining attention scores at each head so that every nuance of the input sequence is taken into consideration. In additive attention (Bahdanau attention mechanism), generative model 112 may make use of attention alignment scores based on a number of factors. Alignment scores may be calculated at different points in a neural network, and/or at different stages represented by discrete neural networks. Source or input sequence words are correlated with target or output sequence words but not to an exact degree. This correlation may consider all hidden states and the final alignment score is the summation of the matrix of alignment scores. In global attention (Luong mechanism), in situations where neural machine translations are required, generative model 112 such as LLM may either attend to all source words or predict the target sentence, thereby attending to a smaller subset of words.

With continued reference to FIG. 1, multi-headed attention in encoder may apply a specific attention mechanism called self-attention. Self-attention allows generative model 112 such as an LLM or components thereof to associate each word in the input, to other words. As a non-limiting example, an LLM may learn to associate the word “you,” with “how” and “are.” It is also possible that an LLM learns that words structured in this pattern are typically a question and to respond appropriately. In some embodiments, to achieve self-attention, input may be fed into three distinct fully connected neural network layers to create query, key, and value vectors. Query, key, and value vectors may be fed through a linear layer; then, the query and key vectors may be multiplied using dot product matrix multiplication in order to produce a score matrix. The score matrix may determine the amount of focus for a word should be put on other words (thus, each word may be a score that corresponds to other words in the time-step). The values in score matrix may be scaled down. As a non-limiting example, score matrix may be divided by the square root of the dimension of the query and key vectors. In some embodiments, the softmax of the scaled scores in score matrix may be taken. The output of this softmax function may be called the attention weights. Attention weights may be multiplied by your value vector to obtain an output vector. The output vector may then be fed through a final linear layer.

With continued reference to FIG. 1, in order to use self-attention in a multi-headed attention computation, query, key, and value may be split into N vectors before applying self-attention. Each self-attention process may be called a “head.” Each head may produce an output vector and each output vector from each head may be concatenated into a single vector. This single vector may then be fed through the final linear layer discussed above. In theory, each head can learn something different from the input, therefore giving the encoder model more representation power. In some cases, encoder of transformer may include a residual connection. Residual connection may include adding the output from multi-headed attention to the positional input embedding. In some embodiments, the output from residual connection may go through a layer normalization. In some embodiments, the normalized residual output may be projected through a pointwise feed-forward network for further processing. The pointwise feed-forward network may include a couple of linear layers with a ReLU activation in between. The output may then be added to the input of the pointwise feed-forward network and further normalized.

With continued reference to FIG. 1, in some cases, decoder component may include a multi-headed attention layer, a pointwise feed-forward layer, one or more residual connections, and layer normalization (particularly after each sub-layer), as discussed in more detail above. In some embodiments, decoder may include two multi-headed attention layers. In some embodiments, decoder may be autoregressive. For the purposes of this disclosure, “autoregressive” means that the decoder takes in a list of previous outputs as inputs along with encoder outputs containing attention information from the input. In some embodiments, input to decoder may go through an embedding layer and positional encoding layer in order to obtain positional embeddings. Decoder may include a first multi-headed attention layer, wherein the first multi-headed attention layer may receive positional embeddings.

With continued reference to FIG. 1, first multi-headed attention layer may be configured to not condition to future tokens. As a non-limiting example, when computing attention scores on the word “am,” decoder should not have access to the word “fine” in “I am fine,” because that word is a future word that was generated after. The word “am” should only have access to itself and the words before it. In some embodiments, this may be accomplished by implementing a look-ahead mask. Look ahead mask is a matrix of the same dimensions as the scaled attention score matrix that is filled with “0s” and negative infinities. For example, the top right triangle portion of look-ahead mask may be filled with negative infinities. Look-ahead mask may be added to scaled attention score matrix to obtain a masked score matrix. Masked score matrix may include scaled attention scores in the lower-left triangle of the matrix and negative infinities in the upper-right triangle of the matrix. Then, when the softmax of this matrix is taken, the negative infinities will be zeroed out; this leaves zero attention scores for “future tokens.” Second multi-headed attention layer may use encoder outputs as queries and keys and the outputs from the first multi-headed attention layer as values. This process matches the encoder's input to the decoder's input, allowing the decoder to decide which encoder input is relevant to put a focus on. The output from second multi-headed attention layer may be fed through a pointwise feedforward layer for further processing.

With continued reference to FIG. 1, the output of the pointwise feedforward layer may be fed through a final linear layer. This final linear layer may act as a classifier. This classifier may be as big as the number of classes that you have. For example, if you have 10,000 classes for 10,000 words, the output of that classifier will be of size 10,000. The output of this classifier may be fed into a softmax layer which may serve to produce probability scores between zero and one. The index may be taken of the highest probability score in order to determine a predicted word. Decoder may take this output and add it to the decoder inputs. Decoder may continue decoding until a token is predicted. Decoder may stop decoding once it predicts an end token. In some embodiment, decoder may be stacked N layers high, with each layer taking in inputs from the encoder and layers before it. Stacking layers may allow an LLM to learn to extract and focus on different combinations of attention from its attention heads.

With continued reference to FIG. 1, as another non-limiting example, generative model 112 may include a generative adversarial network (GAN). As used in this disclosure, a “generative adversarial network” is a type of artificial neural network with at least two sub models (e.g., neural networks), a generator, and a discriminator, that compete against each other in a process that ultimately results in the generator learning to generate new data samples, wherein the “generator” is a component of the GAN that learns to create hypothetical data by incorporating feedbacks from the “discriminator” configured to distinguish real data from the hypothetical data. In some cases, generator may learn to make discriminator classify its output as real. In an embodiment, discriminator may include a supervised machine learning model while generator may include an unsupervised machine learning model as described in further detail below. In an embodiment, discriminator may include one or more discriminative models, i.e., models of conditional probability P(Y|X=x) of target variable Y, given observed variable X. In an embodiment, discriminative models may learn boundaries between classes or labels in given training data. In a non-limiting example, discriminator may include one or more classifiers to distinguish between different categories e.g., “real/related” or “fake/unrelated,” or states e.g., TRUE vs. FALSE within the context of generated data such as, without limitations, set of diagnostic hypotheses as described below, and/or the like. In some cases, processor 104 may implement one or more classification algorithms such as, without limitation, Support Vector Machines (SVM), Logistic Regression, Decision Trees, and/or the like to define decision boundaries. For instance, without limitation, generator of GAN may be responsible for creating synthetic data that resembles real training examples while the discriminator of GAN may evaluate the authenticity of the synthetic data by comparing it to the ground truth. Discriminator may distinguish between genuine and generated content and provide feedback to generator to improve the model performance. Other exemplary embodiment of generative model 112 may include, without limitation, an autoencoder for dimensionality reduction and feature learning, a diffusion model for generating image or audio data, among others. With continued reference to FIG. 1, processor 104 is configured to generate a set of diagnostic hypotheses 124 using generative model 112. As used in this disclosure, a “diagnostic hypothesis” is a tentative identification, prediction, association, or relation to a medical condition, medical disease, cohort, at least an inclusion criteria, or at least an exclusion criteria. In one embodiment, each diagnostic hypothesis within set of diagnostic hypotheses 124 may include information related to a conjectural condition or disease potential be present or develop in an individual e.g., a patient, derived from extrapolation and analysis of corpus 116 containing set of medical literatures as described above. As a non-limiting example, each diagnostic hypothesis within set of diagnostic hypotheses 124 may correlate one or more specific medical conditions with potential diagnostic indicators or patterns recognized within the scope of medical knowledge established during the training of generative model 112. In some cases, processor 104 may be configured to precompute set of hypotheses 124 prior to any processing step as described herein.

With continued reference to FIG. 1, in one or more embodiments, each diagnostic hypothesis may include a data structure encapsulating a plurality of data elements or properties. In one embodiment, generating set of diagnostic hypotheses 124 includes creating a plurality of labels 128, wherein each label of the plurality of labels represents at least one diagnostic feature 132 associated with one or more diagnostic hypotheses within set of diagnostic hypotheses 124. As described herein, a “label” is a categorical marker used to classify and organize data. In some cases, label may be used within generative model 112. In some cases, each label within plurality of labels 128 may represent a specific a conceptual entity that corresponds to a specific aspect or manifestation of medical condition as derived from set of corpus 116. A “diagnostic feature,” for the purpose of this disclosure, is a characteristic or attribute extracted from corpus 116. In one embodiment, at least one diagnostic feature 132 may associated with one or more specific disease or conditions. As a non-limiting example, diagnostic features may include key indicators, symptoms, and/or patterns that are historically or statistically linked to one or more health issues that are gleaned from the analysis of texts, studies, case reports, and/or any medical literatures as described herein.

With continued reference to FIG. 1, as a non-limiting example, set of diagnostic hypotheses may include a first diagnostic hypothesis suggesting a pattern of thickened heart muscle, particularly the spectrum between the ventricles, indicative of hypertrophic cardiomyopathy (HCM) could be present, wherein the first diagnostic hypothesis may be based on diagnostic feature such as genetic markers and echocardiogram (ECG) findings discussed in one or more medical literature in cardiology. As another non-limiting example, set of diagnostic hypotheses may include a second diagnostic hypothesis indicating a likelihood of type 2 diabetes mellitus (T2DM) in a patent, associated with diagnostic feature such as elevated fasting glucose levels, HbA1c percentages, insulin resistance indicators, and/or the like outlined in one or more clinical studies. As a further non-limiting example, set of diagnostic hypotheses may include a third diagnostic hypothesis suggesting chronic obstructive pulmonary disease (COPD) based on a combination of diagnostic features including chronic cough, history of smoking, spirometry results, imaging finding such as hyperinflation or emphysema observed on the chest X-ray or CT scan as documented in a pulmonary research.

With continued reference to FIG. 1, in some cases, creating plurality of labels 128 may include classifying, using a cohort classifier, each diagnostic hypotheses within set of diagnostic hypotheses 124 into one or more labels of plurality of labels 128, and creating plurality of labels 128 as a function of the classification. As used in this disclosure, a “cohort classifier” is a classifier configured to group or segments set of diagnostic hypotheses 124 into one or more distinct categories or labels 128 based on shared characteristics, patterns, or criteria derived from corpus 116. In one or more embodiments, processor 104 may be configured to employ cohort classifier to analyze set of diagnostic hypotheses 124 for patterns that correspond to different disease categories such as, without limitation, cardiovascular diseases, metabolic disorders, neurological conditions, among others. In some cases, cohort classifier may utilize one or more machine-learning techniques as described herein to identifier and group diagnostic hypotheses based on semantic similarity, prevalence data, symptom overlap, and other relevant factors draw from set of medical literatures. As a non-limiting example, generative model 112 may include one or more models of the joint probability distribution P(X, Y) on a given observable variable x, representing features or data that can be directly measured or observed (e.g., set of diagnostic hypotheses 124) and target variable y, representing the outcomes or labels that generative model 112 aims to predict or generate (plurality of labels 128). In some cases, generative model 112 may rely on Bayes theorem to find joint probability; for instance, and without limitation, a Naïve Bayes classifiers may be implemented by processor to categorize set of diagnostic hypotheses 124 into different labels, each represent at least one diagnostic feature 132.

With continued reference to FIG. 1, as a non-limiting example, cohort classifier may include a Naïve Bayes classifier generated, by processor 104, using a Naïve bayes classification algorithm. Naïve Bayes classification algorithm generates classifiers by assigning class labels to problem instances, represented as vectors of element values. Class labels are drawn from a finite set. Naïve Bayes classification algorithm may include generating a family of algorithms that assume that the value of a particular element is independent of the value of any other element, given a class variable. Naïve Bayes classification algorithm may be based on Bayes Theorem expressed as P(A/B)=P(B/A) P(A)÷P(B), where P(A/B) is the probability of hypothesis A given data B also known as posterior probability; P(B/A) is the probability of data B given that the hypothesis A was true; P(A) is the probability of hypothesis A being true regardless of data also known as prior probability of A; and P(B) is the probability of the data regardless of the hypothesis. A naïve Bayes algorithm may be generated by first transforming training data into a frequency table. Processor 104 may then calculate a likelihood table by calculating probabilities of different data entries and classification labels. Processor 104 may utilize a naïve Bayes equation to calculate a posterior probability for each class. A class containing the highest posterior probability is the outcome of prediction.

With continued reference to FIG. 1, processor 104 is configured to receive biomedical signal 136 pertaining to a patient 140. As used in this disclosure, a “biomedical signal” refers to any type of signal, data, or information that captures physiological activity, phenomena, or characteristics of a living organism. In some cases, biomedical signal 136 may be received through one or more input devices. “Input device” for the purposes of this disclosure is a device capable of transmitting information to processor 104. Exemplary input device may include a keyboard, a mouse, a touchscreen, a smartphone, a network server, a sensor and/or the like. As a non-limiting example, input devices may include a medical device and sensors designed to detect and record electrical thermal, mechanical, and/or chemical changes associated with bodily functions and conditions of a human. In one or more embodiments, reception of biomedical signal 136 may include systematic acquisition processing of data signal generated from one or more medical devices and sensors that monitor or measure physiological parameters of patient 140. Exemplary biomedical signals 136 may include, without limitation, ECG data, magnetic resonance imaging (MRI) scans, computed tomography (CT_scans), and the like as described in further detail below.

With continued reference to FIG. 1, as used in this disclosure, a “signal” is any intelligible representation of data, for example from one device to another. In some cases, a signal may be used to communicate with apparatus 100, for example by way of one or more ports. In some cases, a signal may be transmitted and/or received by a computing device for example by way of an input/output port. An analog signal may be digitized, for example by way of an analog to digital converter. In some cases, an analog signal may be processed, for example by way of any analog signal processing steps described in this disclosure, prior to digitization. In some cases, a digital signal may be used to communicate between two or more devices, including without limitation computing devices. In some cases, a digital signal may be communicated by way of one or more communication protocols, including without limitation internet protocol (IP), controller area network (CAN) protocols, serial communication protocols (e.g., universal asynchronous receiver-transmitter [UART]), parallel communication protocols (e.g., IEEE 128 [printer port]), and the like.

With continued reference to FIG. 1, in some cases, processor 104 may perform one or more signal processing steps on a signal. For instance, apparatus 100 may analyze, modify, and/or synthesize a signal representative of data in order to improve the signal, for instance by improving transmission, storage efficiency, or signal to noise ratio. Exemplary methods of signal processing may include analog, continuous time, discrete, digital, nonlinear, and statistical. Analog signal processing may be performed on non-digitized or analog signals. Exemplary analog processes may include passive filters, active filters, additive mixers, integrators, delay lines, compandors, multipliers, voltage-controlled filters, voltage-controlled oscillators, and phase-locked loops. Continuous-time signal processing may be used, in some cases, to process signals which varying continuously within a domain, for instance time. Exemplary non-limiting continuous time processes may include time domain processing, frequency domain processing (Fourier transform), and complex frequency domain processing. Discrete time signal processing may be used when a signal is sampled non-continuously or at discrete time intervals (i.e., quantized in time). Analog discrete-time signal processing may process a signal using the following exemplary circuits sample and hold circuits, analog time-division multiplexers, analog delay lines and analog feedback shift registers. Digital signal processing may be used to process digitized discrete-time sampled signals. Commonly, digital signal processing may be performed by a computing device or other specialized digital circuits, such as without limitation an application specific integrated circuit (ASIC), a field-programmable gate array (FPGA), or a specialized digital signal processor (DSP). Digital signal processing may be used to perform any combination of typical arithmetical operations, including fixed-point and floating-point, real-valued, and complex-valued, multiplication and addition. Digital signal processing may additionally operate circular buffers and lookup tables. Further non-limiting examples of algorithms that may be performed according to digital signal processing techniques include fast Fourier transform (FFT), finite impulse response (FIR) filter, infinite impulse response (IIR) filter, and adaptive filters such as the Wiener and Kalman filters. Statistical signal processing may be used to process a signal as a random function (i.e., a stochastic process), utilizing statistical properties. For instance, in some embodiments, a signal may be modeled with a probability distribution indicating noise, which then may be used to reduce noise in a processed signal.

With continued reference to FIG. 1, as a non-limiting example, biomedical signal 136 may include ECG data pertaining to patient 140. “Electrocardiogram data” for the purposes of this disclosure, is information associated with electrocardiogram signals. In one or more embodiments, electrocardiogram data may include a matrix (i.e., an array of numbers arranged in rows or columns) having a plurality of electrocardiogram signals and/or associated a plurality of timestamps. As used in the current disclosure, a “electrocardiogram signal” is a signal representative of electrical activity of a heart. “ECG data” may be used interchangeably with electrocardiogram signal within this disclosure. In one or more embodiments, ECG signals may be received by one or more electrodes connected to the skin of patient 140. In one or more embodiments, ECG signals may represent depolarization and repolarization occurring in the heart. In one or more embodiments, ECG signals may be captured periodically. For example, and without limitation, every second, every millisecond and the like. In some cases, plurality of timestamps may increase in given increments, such as for example, in increments of 5 ms, wherein a first time timestamp may include 5 ms and a second timestamp may include 10 ms. In one or more embodiments, a combination of a plurality of ECG signals and correlated timestamps may be used to generate a graph illustrating the heart functions of an individual, also known as an “ECG image.” In one or more embodiments, processor 104 may be configured to receive one or more ECG images pertaining to patient 140. Additionally, or alternatively, ECG signals may be captured as voltages, such as millivolts or microvolts.

With continued reference to FIG. 1, in some cases, processor 104 may be configured to receive biomedical signal such as ECG data from one or more sensors. As used in this disclosure, a “sensor” is a device that is configured to detect an input and/or a phenomenon and transmit information related to the detection. In one embodiment, sensor may detect a plurality of data including, without limitation, electrocardiogram signals, heart rate, blood pressure, electrical signals related to the heart, timestamps associated with captured data and the like. In some cases, sensor may include one or more electrodes. Electrodes used for an electrocardiogram (ECG) are conductive patches that are placed on specific locations on the body of patient 140 to detect and record the electrical signals generated by the heart. Senor may serve as the interface between patient's 140 body and the ECG machine, allowing for the measurement and recording of the heart's electrical activity. As a non-limiting example, 10 electrodes may be used for a standard 12-lead ECG, placed in specific positions on the chest and limbs of the patient. Electrodes are typically made of a conductive material, such as metal or carbon, and are connected to lead wires that transmit the electrical signals to the ECG machine for recording. In one or more embodiments, ECG data may include a 12-lead electrocardiogram. In some cases, sensors may include wireless sensors wherein data may be received from sensor and transmitted to processor 104 wirelessly. In one or more embodiments, wireless sensors may include Bluetooth enabled ECG sensors, RFID ECG sensors, Wi-Fi enabled ECG sensors and the like. In one or more embodiments, wireless sensors may allow for receipt of data from a distance. In one or more embodiments, wireless sensors may allow for a machine or system to receive data without wires connecting the sensors to processor 104. In one or more embodiments, the presence of wires from sensors to processor 104 may obstruct medical personnel from conducting one or more medical treatment procedures.

With continued reference to FIG. 1, one or more sensors may be placed on each limb, wherein there may be at least one sensor on each arm and leg. These sensors may be labeled I, II, III, V1, V2, V3, V4, V5, V6, and the like. For example, Sensor I may be placed on the left arm, Sensor II may be placed on the right arm, and Sensor III may be placed on the left leg. Additionally, a plurality of sensors may be placed on various portions of the patient's torso and chest. For example, a sensor V1 may be placed in the fourth intercostal space at both the right sternal borders and sensor V2 may be fourth intercostal space at both the left sternal borders. A sensor V3 may also be placed between sensors V2 and V4, halfway between their positions. Sensor V4 may be placed in the fifth intercostal space at the midclavicular line. Sensor V5 may be placed horizontally at the same level as sensor V4 but in the anterior axillary line. Sensor V6 may be placed horizontally at the same level as V4 and V5 but in the midaxillary line. In one or more embodiments, each sensor and/or lead may contain a set of electrical signals, wherein ECG data as described herein may include ECG signals associated with each lead and/or sensor.

With continued reference to FIG. 1, in some cases, one or more sensors may include augmented unipolar sensors. These sensors may be labeled as aVR, aVL, and aVF. These sensors may be derived from the limb sensors and provide additional information about the heart's electrical activity. These leads are calculated using specific combinations of the limb leads and help assess the electrical vectors in different orientations. For example, aVR may be derived from Sensor II and Sensor III. In another example, aVL may be derived from sensor I and Sensor III. Additionally, aVF may be derived from Lead I and Lead II. The combination of limb sensors, precordial sensors, and augmented unipolar sensors allows for a comprehensive assessment of the heart's electrical activity in three dimensions. These leads capture the electrical signals from different orientations, which are then transformed into transformed coordinates to generate vectorcardiogram (VCG) representing magnitude and direction of electrical vectors during cardiac depolarization and repolarization. Transformed coordinates may include one or more a Cartesian coordinate system (x, y, z), polar coordinate system (r, θ), cylindrical coordinate system (ρ, φ, z), or spherical coordinate system (r, θ, φ). In some cases, transformed coordinates may include an angle, such as with polar coordinates, cylindrical coordinates, and spherical coordinates. In some cases, VCG may be normalized thus permitting full representation with only angles, i.e., angle traversals. In some cases, angle traversals may be advantageously processed with one or more processes, such as those described below and/or spectral analysis.

With continued reference to FIG. 1, in one or more embodiments, sensor may include surface electrodes wherein the surface electrodes may be placed above the skin of a user and used to detect electrical impulses. In one or more embodiments, sensor may further include a wearable ECG monitor wherein the wearable ECG monitor may be wrapped around a limb of the individual and used to detect electrical impulses. In one or more embodiments, sensor may further include a Holter monitor, subdermal needle electrodes, and/or any other sensing device capable of receiving electrical signals. As a non-limiting example, biomedical signal 136 may include a plurality of ECG signals 124 captured at discrete time intervals in a digital imaging and communications in medicine (DICOM) Format, a CSV format, as a spread sheet containing cells for each datum and the like. In one or more embodiments, processor 104 may receive data in a raw format wherein the data may be converted into ECG data represented as a matrix as described above.

With continued reference to FIG. 1, in some cases, processor 104 may be configured to receive biomedical signal 136 or subsequently convert biomedical signal 136 into a textual format. A “Textual format” for the purposes of this disclosure is a format in which a set of data is represented by characters, numbers, or any other alphanumeric representations. As a non-limiting example, a set of data may be said to be in textual format in instances in which the contents of the file contain only characters of readable material. In one or more embodiments, data in textual format may be contrasted with an image, video and the like. In one or more embodiments, data within a textual format may include machine-readable alphanumeric characters. In one embodiments, biomedical signal 136 may include an electronic file, such as .txt, .docx, .xlsx, or the like containing ECG data in a textural format. In such embodiment, ECG data may include textual data corresponding to Leads and corresponding voltage signals of the leads. As a non-limiting example, generative model 112 such as a LLM may receive an input. Input may include a textural input, for example, a string of one or more characters, words, sentences, paragraphs, queries describing ECG data. A “query” for the purposes of the disclosure is a string of characters that poses a question. In some cases, such input may be received from a user device. User device may be any computing device that is used by a user. As non-limiting examples, user device may include desktops, laptops, smartphones, tablets, and the like. In one embodiment, receiving biomedical signal 136 may include receiving ECG data as a prompt to LLM.

With continued reference to FIG. 1, additionally, or alternatively, processor 104 may be configured to receive biomedical signal 136 in an image format. In one or more embodiments, biomedical signal 136 may include imaging signal such as, without limitation, MRI, CT scans, X-rays, and/or any other images providing visual insights into internal structures of patient's 140 body. As a non-limiting example, processor 104 may be configured to receive one or more ECG images pertaining to patient 140. In some cases, biomedical signal 136 may include standardized data (transformed from raw ECG images) in consistent with U.S. patent application Ser. No. 18/641,217, filed on Apr. 19, 2024, and entitled “SYSTEMS AND METHODS FOR TRANSFORMING ELECTROCARDIOGRAM IMAGES FOR USE IN ONE OR MORE MACHINE LEARNING MODELS,” wherein its entirety is incorporate herein by reference. Further, processor 104 may be configured to receive biomedical signal 136 in audio format; for instance, and without limitation, acoustic signal such as, without limitation, heart sounds, lung sounds (recorded during spirometry), vocal patterns, and/or the like. As a person skilled in the art, will be aware of the necessity to employ specific models or sub models such as, without limitation, convolution neural networks (CNNs), RNNs, long short-term memory network (LSTM), among others within generative model 112 tailored to efficiently process and analyze various input data modality listed above. In some cases, generative model 112 may be configured to integrate and switch between a plurality of specialized models or sub-models based on different types of biomedical signal 136 in different format.

With continued reference to FIG. 1, in some cases, biomedical signals 136 may be received from database 120 communicatively connected to processor 104 as described above. In one embodiment, database 120 may include a repository of historical and anonymized patient ECG recordings. In some cases, biomedical signals 136 may include real-time streaming data. In one embodiment, processor 104 may be in communication with one or more continuous monitoring devices such as wearable heart rate monitors or continuous glucose monitoring systems that generate real-time streaming data reflecting patient's 140 physiological state over time. In such embodiment, processor 104 and/or generative model 112 may receive sequential data input in real-time or near-real time. In other cases, biomedical signals 136 may be received from external platforms e.g., 3^{rd} party telehealth platforms or other remote patient monitoring services, where biomedical signals 136 may be transmitted securely over the internet and the cloud.

With continued reference to FIG. 1, processor 104 is configured to identify at least one biomedical feature 144 as a function of biomedical signal 136. As used in this disclosure, a “biomedical feature” is a attribute, characteristic, or otherwise a marker within a biomedical signal. In some cases, biomedical feature may provide information regarding physiological or pathological state of a patient. In one embodiment, biomedical feature 144 may include an ECG feature identified from ECG data as described above, wherein the “ECG feature,” for the purpose of this disclosure, is a characteristic or attribute derived from ECG data.” ECG feature may be quantifiable. ECG feature may provide information regarding electrical activity and functioning of patient's heart. Exemplary ECG feature may include, without limitation, heart rate, PR interval, QT internal, ST segment, and/or the like. As another non-limiting example, biomedical features 144 such as an ECG features may include several distinct waves and intervals, each representing a different phase of the cardiac cycle, such as P-wave, QRS complex, T wave, U wave, and the like. The P-wave may represent atrial depolarization (contraction) as the electrical impulse spreads through the atria. The QRS complex may represent ventricular depolarization (contraction) as the electrical impulse spreads through the ventricles. The QRS complex may include three waves: Q wave, R wave, and S wave. The T-wave may represent ventricular repolarization (recovery) as the ventricles prepare for the next contraction. The U-wave may sometimes be present after the T wave, it represents repolarization of the Purkinje fibers. The intervals between these waves may provide information about the duration and regularity of various phases of the cardiac cycle. Other exemplary biomedical features may include, without limitation, brain wave patterns, tumor markers in MRI/CT images, blood glucose levels, and/or the like.

With continued reference to FIG. 1, at least one biomedical feature 144 such as ECG feature may include at least one data element describing a cardiac abnormality 148. As used in this disclosure, a “cardiac abnormality” is any deviation or irregularity in associated with or pertaining with the heart, for example the heart's structure, function, relationship with other organs or body parts, biological activity, or electrical activity that differs from established normal parameters. In some cases, cardiac abnormality may manifest in various forms including, but not limited to, arrhythmias (abnormal heart rhythms), ischemic changes (indications of reduced blood flow to the heart muscle), structural defects, electrical conduction issues, and/or the like. In some cases, biomedical features 144 may include features a medical professional typically notice. As a non-limiting example, data element describing cardiac abnormality 148 may include a data element that specifies the length of QT interval in milliseconds. A QT internal that exceeds a pre-determined range may indicate a long QT syndrome which is a risk factor for Torsades de Pointes (i.e., a type of arrhythmia). In other cases, biomedical features 144 may also include subtle features not ordinarily noticed by medical professional without advanced analysis. As a non-limiting example, data element describing cardiac abnormality 148 may include a data element quantifies an elevation level of ST segment from a baseline indicative of ST-segment elevation myocardial infarction (STEMI). As another non-limiting example, data element describing cardiac abnormality 148 may identify and quantify instances where the T wave is inverted in specific leads where it is normally upright. As a further non-limiting example, data element describing cardiac abnormality 148 may include a minor variations in morphology of the QRS complex. Additionally, or alternatively, at least one data element describing cardiac abnormality 148 may include a value used to indicate a level of atrial fibrillation, tachycardia, premature beats, bradycardia, heart block, heart palpitations, and/or the like. In one embodiment, at least one data element describing cardiac abnormality 148 may further include an ejection fraction level. As a non-limiting example, at least one data element describing cardiac abnormality 148 may include a cardiac value, wherein the “cardiac value,” as used herein, is information associated with a heart disease or heart condition. For instance, data element may include an ejection fraction of 50%.

With continued reference to FIG. 1, processor 104 may be configured to perform one or more feature extraction algorithms to identify at least one biomedical feature 144 from biomedical signal 136. In some embodiments, one or more feature extraction algorithms may be designed to isolate and quantify specific characteristics or markers from biomedical signals 136. In some cases, feature extraction algorithms may include model-based approaches; for instance, and without limitation, at least one biomedical feature 144 may be identified, at generative model 112, as a function of biomedical signal 136, wherein generative model 112 may include one or more models configured to extract detailed spatial hierarchies from the received imaging signal. As a non-limiting example, one or more convolution neural networks (CNNs) may be employed to process imaging signal. In some cases, receiving biomedical signal 136 may include receiving MRI or CT scan sequences including temporal dimensions (e.g., functional MRI or fMRI) at CNNs. In some cases, CNNs may include 3D CNNs or CNNs combined with RNN. Additionally, or alternatively, at least one biomedical feature 144 may be predicted based on biomedical signals. One or more machine learning models may be trained on example biomedical signals and associated example biomedical features to predict at least one biomedical feature 144 in the absence of explicit feature extraction. In some cases, one or more feature learning algorithms (i.e., unsupervised learning) such as clustering algorithms may be applied to biomedical signal 136. Processor 104 may be configured to predict at least one biomedical feature 144 as a function of biomedical signal 136 using the trained models upon receipt of the biomedical signal 136. As a non-limiting example, generative model 112 may include a deep learning model trained on ECG signals may be configured to predict ECG feature such as heart rate variability (HRV) or the presence of arrhythmias without needing to extract these biomedical features from the ECG data. In some cases, biomedical feature 144 such as ECG feature may be extracted through one or more ECG machine learning models trained to classify, for example and without limitation, classify a patient's ejection fraction into multiple categories such as “normal,” “mildly abnormal,” “moderately abnormal,” and “severely abnormal.”

With continued reference to FIG. 1, in one or more embodiments, processor 104 may be configured to identify a plurality of biomedical features as a function of biomedical signal 136. In some cases, plurality of biomedical features may be evaluated based on a set of pre-determined criteria to ascertain, for example, their clinical significance or relevance in the context of patient diagnosis and healthcare. In one embodiment, set of pre-determined criteria may be used to differentiate between clinically significant abnormalities that necessitate medical intervention and minor anomalies that may not impact patient's health. As a non-limiting example, processor 104 may be configured to filter plurality of ECG features according to one or more pre-determined criteria. In some cases, biomedical features 144 that significantly deviate from established normal ranges for patient's 140 demographic (age, gender, etc.,), identification of biomedical features 144 that correspond to known medical conditions or risk factors, biomedical features 144 persist over multiple readings or show a consistent trend over time, biomedical features 144 that correlate with patient-reported symptoms, biomedical features 144 indicating conditions with severe outcome or higher risk of progression, biomedical features 144 that influence treatment decisions (including choice of medication, necessity for surgery, other medical inventions etc.,) and the like may be flagged.

With continued reference to FIG. 1, in one or more embodiments, identifying at least one biomedical feature 144 may include extracting a plurality of ECG features from ECG data, ranking the plurality of ECG features based on set of pre-determined criteria, and identifying the at least one ECG feature from the plurality of ECG features based on the rank of the plurality of ECG features. As a non-limiting example, plurality of ECG features may be processed and filtered, as described above, based on one or more pre-determined criteria selected from set of pre-determined criteria according to clinical urgency, diagnostic value, prognostic significance, patient-specific context, symptom frequency and consistency, or any combination thereof. In some cases, processor 104 may implement a specialized ranking algorithm configured to apply one or more pre-determined criteria to each ECG feature of plurality of ECG features. As a non-limiting example, such ranking algorithm may use weighted factors for each criterion based on its clinical importance. In some cases, ranking algorithm may also be configured to adjust calculated ranks based on inter-feature relationships i.e., how presence of one ECG feature may influence the significance of another ECG feature. In one embodiment, ranking plurality of ECG features may include prioritizing plurality of ECG features with ECG features ranked highest based on selected criteria at the top in a prioritized data structure e.g., a prioritized list. Processor 104 may be configured to select, form prioritized list, ECG features above certain threshold for further processing as described below.

With continued reference to FIG. 1, processor 104 is configured to select at least one diagnostic hypothesis 152 from set of diagnostic hypotheses 148 for patient 140 by matching at least one biomedical feature 144 against at least one diagnostic feature 132. In one embodiment, at least one diagnostic hypothesis 152 may include a most reasonable and educated guess or predictions about one or more possible medical conditions or diseases that patient 140 may have, based on biomedical signal 136 such as, without limitation, ECG data as described above. In some cases, matching at least one biomedical feature 144 against at least one diagnostic feature 132 may include comparing, using processor 104, each biomedical feature identified from patient's 140 biomedical signal 136 to each diagnostic feature associated with plurality of labels 128 derived from corpus 116 and encoded within generative model's 112 knowledge base. At least one diagnostic hypothesis 152 may be selected from set of diagnostic hypotheses 148 generated, by generative model 112, prior to the receipt of patient's 140 biomedical signal 136 based on the match. In some cases, processor 104 may iterate through set of diagnostic hypotheses and select at least one diagnostic hypothesis 152 upon a positive match. In some cases, processor 104 may select a plurality of diagnostic hypotheses having diagnostic features matched with patient's 140 biomedical features such as ECG features. As a non-limiting example, positive match may be determined based on the similarity in ECG signal pattern, magnitude, frequency, or temporal properties and the like being compared. For instance, a prolonged QT interval as a biomedical feature from an ECG may match a diagnostic feature associated with Long QT Syndrome, among other conditions.

With continued reference to FIG. 1, in one embodiment, both biomedical features and diagnostic features may be transformed into a vector space model. As a non-limiting example, processor 104 may be configured to represent at least one biomedical feature 144 and at least one diagnostic feature 132 as vectors in a high-dimensional space where the dimensions correspond to attributes or characteristics of the feature. A “vector” as defined in this disclosure is a data structure that represents one or more a quantitative values and/or measures of a given feature. A “vector space,” as defined in his disclosure, is a set of mathematical objects that can be added together under an operation of addition following properties of associativity, commutativity, existence of an identity element, and existence of an inverse element for each vector, and can be multiplied by scalar values under an operation of scalar multiplication compatible with field addition, and that has an identity element is distributive with respect to vector addition, and is distributive with respect to field addition. In one embodiment, a vector may be represented as an n-tuple of values, where n is one or more values, as described in further detail below;

With continued reference to FIG. 1, each value of n-tuple of values may represent a measurement or other quantitative value associated with a given category of data, or attribute, examples of which are provided in further detail below; a vector may be represented, without limitation, in n-dimensional space using an axis per category of value represented in n-tuple of values, such that a vector has a geometric direction characterizing the relative quantities of attributes in the n-tuple as compared to each other. Two vectors may be considered equivalent where their directions, and/or the relative quantities of values within each vector as compared to each other, are the same; thus, as a non-limiting example, a vector represented as [5, 10, 15] may be treated as equivalent, for purposes of this disclosure, as a vector represented as [1, 2, 3]. Vectors may be more similar where their directions are more similar, and more different where their directions are more divergent, for instance as measured using cosine similarity as computed using a dot product of two vectors; however, vector similarity may alternatively or additionally be determined using averages of similarities between like attributes, or any other measure of similarity suitable for any n-tuple of values, or aggregation of numerical similarity measures for the purposes of loss functions as described in further detail below.

With continued reference to FIG. 1, any vectors as described herein may be scaled, such that each vector represents each attribute along an equivalent scale of values. As a non-limiting example, processor 104 may be configured to normalize at least one biomedical feature 144 and at least one diagnostic feature 132 to ensure that comparisons are not biased by the scale of measurements or, in other cases, the intensity of signals. Each vector may be “normalized,” or divided by a “length” attribute, such as a length attribute 1 as derived using a Pythagorean norm:

$$l = \sqrt{{\sum }_{i = 0}^{n} {{a}_{i}}^{2}} ,$$

where ai is attribute number i of the vector. Scaling and/or normalization may function to make vector comparison independent of absolute quantities of attributes, while preserving any dependency on similarity of attributes. A two-dimensional subspace of a vector space may be defined by any two orthogonal vectors contained within the vector space. Two-dimensional subspace of a vector space may be defined by any two orthogonal and/or linearly independent vectors contained within the vector space; similarly, an n-dimensional space may be defined by n vectors that are linearly independent and/or orthogonal contained within a vector space. A vector's “norm’ is a scalar value, denoted ∥a∥ indicating the vector's length or size, and may be defined, as a non-limiting example, according to a Euclidean norm for an n-dimensional vector a as:

$$‖ a ‖ = \sqrt{{\sum }_{i = 0}^{n} {a}_{i}^{2}}$$

With continued reference to FIG. 1, in some cases, comparing at least one biomedical feature 144 and at least one diagnostic feature 132 may include comparing, between the two feature vectors, their similarity. In one embodiment, processor may be configured to compute a degree of vector similarity between a vector representing at least one biomedical feature 144 and a vector representing at least one diagnostic feature 132. In some cases, vector similarity may be measured according to any norm for proximity and/or similarity of two vectors, including without limitation cosine similarity. As used in this disclosure “cosine similarity” is a measure of similarity between two-non-zero vectors of a vector space, wherein determining the similarity includes determining the cosine of the angle between the two vectors. Cosine similarity may be computed as a function of using a dot product of the two vectors divided by the lengths of the two vectors, or the dot product of two normalized vectors. For instance, and without limitation, a cosine of 0° is 1, wherein it is less than 1 for any angle in the interval (0,π) radians. Cosine similarity may be a judgment of orientation and not magnitude, wherein two vectors with the same orientation have a cosine similarity of 1, two vectors oriented at 90° relative to each other have a similarity of 0, and two vectors diametrically opposed have a similarity of −1, independent of their magnitude. As a non-limiting example, vectors may be considered similar if parallel to one another, signifying a higher degree of match. As a further non-limiting example, vectors may be considered dissimilar if orthogonal (or even opposite) to one another indicative of a lower degree of match. Additionally, or alternatively, degree of similarity may include any other geometric measure of distance between vectors.

With continued reference to FIG. 1, in some embodiments, selecting the at least one diagnostic hypothesis 152 may additionally, or alternatively, include determining, for each diagnostic hypothesis within set of diagnostic hypotheses, a confidence level 156, and select at least one diagnostic hypothesis 152 as a function of the confidence level 156. As used in this disclosure, a “confidence level” is a probabilistic measure that reflects a likelihood or certainty. For instance, confidence level may indicate a level of certainty that a given hypothesis accurately represents a condition based on the analyzed data. In some case, confidence level 156 may be expressed as a category, a percentage, or otherwise a score. In one embodiment, confidence level 156 may be quantitatively assessed based on the strength of the match between at least one biomedical feature 144 and at least one diagnostic feature 132 associated with each hypothesis as well as the overall relevance and consistency of set of hypotheses 124 with known medical literatures. In some cases, one or more machine learning models may be employed to analyze degree of correlation between at least one biomedical feature 132 and at least one diagnostic feature 132 as described above. As a non-limiting example, a “high,” “100%” or a full (score) confidence level may represent a direct match. In some cases, determination of confidence level 156 may also incorporate one or more clinical inputs, for example, and without limitation, inputs containing information related to prevalence of the condition in similar patient populations or the community, known risk factors present in patient 140, and any corroborating clinical findings (in set of medical literatures) that support the hypothesis. In one or more embodiments, one or more probabilistic models may be implemented, by processor 104, to calculate confidence level 156 for each diagnostic hypothesis within set of diagnostic hypotheses 124 by integrating these clinical inputs into a unified probabilistic framework. As a non-limiting example, Bayesian inference or other statical methods may be used to estimate a probability that the corresponding diagnostic hypothesis is correct. Additionally, or alternatively, a threshold confidence level may be established based on clinical scenario, urgency of diagnosis, and/or the like. As a non-limiting example, diagnostic hypotheses associated with confidence levels above threshold confidence level may be considered sufficiently reliable to be selected for further processing while diagnostic hypotheses associated with confidence levels below threshold confidence level may be considered a false-positive diagnostic hypothesis.

With continued reference to FIG. 1, in other embodiments, selecting the at least one diagnostic hypothesis 152 may include aggregating a plurality of diagnostic hypothesis based on multiple matches. In some cases, apparatus 100 may be configured to consider the collective implication of set of diagnostic hypothesis to formulate at least one diagnostic hypothesis 152. In some cases, once diagnostic hypotheses within set of diagnostic hypotheses 124 are ranked and each diagnostic hypothesis associated with its own confidence level 156, processor 104 may be configured to determine a plurality of diagnostic hypotheses, wherein a combination of the plurality of diagnostic hypotheses may suggest a more complex or multi-faceted medical condition that would not be apparent when considering features singly. As a non-limiting example, at least one diagnostic hypothesis 152 may describe a synergy between different medical conditions such as cooccurrence of diseases that may share similar biomedical features or a manifestation of one condition as a complication of another. In some cases, at least one diagnostic hypothesis 152 may be generated, by processor 104, as a function of plurality of diagnostic hypotheses selected from set of diagnostic hypotheses 124 as a function of the confidence levels. For instance, concurrent suggestion of hypertension and left ventricular hypertrophy in aggregated diagnostic hypotheses may reinforce the likelihood of a more complex cardiac condition requiring an alternative treatment approach.

With continued reference to FIG. 1, processor 104 is configured to query, as a function of at least a matched label 160, a medical repository 164 in communication with processor 104, to validate at least one diagnostic hypothesis 152. As used in this disclosure, a “medical repository” is a collection of medical data and information. Medical repository may be structured, semi-structure, or unstructured. Medical repository may be used for reference. In some cases, medical repository 164 may contain data, such as without limitation, clinical data, research findings, case studies, diagnostic criteria, treatment outcomes, patient records, and/or the like. As a non-limiting examples, medical repository 164 may include a plurality of electronic health records (EHRs) 168 associated with a plurality of patients. “Electronic health records,” for the purpose of this disclosure, are digital versions of patient health records. In some cases, medical repository 164 may include a plurality of real-time, patient-centered records that is available instantly and securely to connected processor 104. In one or more embodiments, each EHR of plurality of EHRs 168 may include demographic information, medical history, medication and allergies information, immunization status, laboratory test results, radiology images, vital signs, personal statistics (e.g., age and weight), billing information, and/or the like. As a non-limiting example, medical repository 164 may include a centralized or distributed source of medical data such as a hospital information system (HIS), regional health information organization (RHIO), health information exchange (HIE), cloud-base EHR platform, research database and biobank, public health database, clinical registry, among others.

With continued reference to FIG. 1, in one or more embodiments, querying medical repository 164 involves processor 104 actively searching and retrieving relevant information from medical repository 164 based on at least a matched label 160. In some cases, at least one matched label may include at least one label represents a diagnostic feature paired with at least one label representing a biomedical feature derived from biomedical signal pertaining to patient 140 as described above. As a non-limiting example, processor 104 may be configured to formulate one or more queries by translating at least a matched label into one or more query terms that are compatible with indexing and organization of medical repository 164. In some cases, at least a matched label 160 may be translated into one or more standardized medical terminology or codes; for instance, and without limitation, international classification of diseases (ICD-10) codes, current procedural terminology (CPT) codes, or logical observation identifiers names and codes (LOINC) for laboratory tests and clinical observations. As another non-limiting example, generative model 112 such as, without limitation, may be configured to generate a query as a function of at least one diagnostic hypothesis 152. In some cases, processor 104 may dynamically construct a query that encompass at least one diagnostic hypothesis 152 and/or related diagnostic hypotheses within set of diagnostic hypotheses 124 containing related conditions, possible complications, and/or associated treatment options. In some cases, constructing query may include convert biomedical feature 144 such as ECG feature into vector embeddings, for instance, if the diagnostic hypothesis suggests “atrial fibrillation (AFib),” generative model 112 may extend query to include vector embeddings describing ECG patterns indicative of AFib, and pull up similar ECGs, from medical repository 164 based on the embeddings.

With continued reference to FIG. 1, in some cases, processor 104 may send one or more query to medical repository 164 and execute the queries to search indices and/or database to find one or more matches or relevant entries for validation. Medical repository 164 may returns information that matches the query criteria, upon execution of the queries, wherein such information may include, without limitation, diagnostic criteria, recent symptoms and treatments, prognostic information, and/or the like. In one or more embodiment, validating at least one diagnostic hypothesis 152 may include retrieving, from medical repository 164, one or more EHRs from plurality of EHRs 168 as a function of at least a matched label 160 and comparing at least one diagnostic hypothesis 152 with one or more reference biomedical signals 172 encapsulated in the one or more EHRs. As used in this disclosure, “reference biomedical signals” are biomedical signals that are used for reference, e.g., relational comparisons with other biomedical signals. In some cases, reference biomedical signals may include previously recorded or standardized biomedical signal that are stored within EHRs and serve as benchmarks or comparative baselines for interpreting new patient data. As a non-limiting example, reference biomedical signals 172 may include one or more standardized (and verified) ECG tracings, patterns, and/or the like. In some cases, comparing biomedical signal with reference biomedical signals may include, for instance, comparing and correlating at least one diagnostic hypothesis 152 with existing medical knowledge and evidence associated with patient 140 found in medical repository 164. Such validation of at least one diagnostic hypothesis 132 may ensure it is supported by credible medical data and aligns with existing patterns of condition it may suggest. In some cases, medical repository 164 may be at least a portion of database 120 as described above, separate from corpus 116.

With continued reference to FIG. 1, in some cases, at least one diagnostic hypothesis 152 may be validated if and only if the information retrieved from medical repository 164 includes one or more diagnostic features (or criteria) associated with at least one diagnostic hypothesis 152 that match patient's 140 biomedical features. In one embodiment, at least one diagnostic hypothesis 152 may gain validity if medical repository 164 provides one or more case studies or clinical reports of patients with similar features who were diagnosed with condition suggested by at least one diagnostic hypothesis 152. In some cases, patients may include other patients in different community having biomedical signals or EHRs similar to patient 140. In another embodiment, at least one diagnostic hypothesis 152 may be further validated by retrieving and analyzing research articles, clinical trial results, or guidelines that support connection between identified biomedical feature 144 and suggested diagnosis. In some cases, validating at least one diagnostic hypothesis 152 may include citing one or more medical literatures within set of medical literatures within corpus 116 as described above. In other embodiments, processor 104 may be configured to check consistency between at least one diagnostic hypothesis 152 and relevant patient history or related conditions documented within patient's 140 EHR. Additionally, or alternatively, query result e.g., reference biomedical signals may be sued to refine or adjust at least one diagnostic hypothesis. In some cases, refining or adjusting at least one diagnostic hypothesis may be prior to the comparison thereby improving apparatus 100 precision and accuracy.

With continued reference to FIG. 1, an LLM may generate at least one annotation as an output. At least one annotation may be any annotation as described herein. In some embodiments, an LLM may include multiple sets of transformer architecture as described above. Output may include a textual output. A “textual output,” for the purposes of this disclosure is an output comprising a string of one or more characters. Textual output may include, for example, a plurality of annotations for unstructured data. In some embodiments, textual output may include a phrase or sentence identifying the status of a user query. In some embodiments, textual output may include a sentence or plurality of sentences describing a response to a user query. As a non-limiting example, this may include restrictions, timing, advice, dangers, benefits, and the like. It should be noted that LLM may generate outputs in any data modalities as described herein such as, without limitation, images, audios, videos, and/or the like.

With continued reference to FIG. 1, processor 104 may be configured to output at least one diagnostic hypothesis 152 upon a positive validation of at least one diagnostic hypothesis 152. In one or more embodiments, outputting at least one diagnostic hypothesis 152 may include outputting at least one diagnostic hypothesis 152, at generative model 112 such as an LLM, the at least one diagnostic hypothesis as a function of the comparison as described above in a remote configuration. In other embodiments, at least one diagnostic hypothesis 152 may be directly output to one or more requesting entities (i.e., any induvial, group, organization, device, system, system/device component, or application that request access to or delivery of at least one diagnostic hypothesis 152) in a local set up. As a non-limiting example, at least one diagnostic hypothesis 152 may be transmitted, upon a positive validation, to patient 140 and or one or more physicians or other medical professionals working with patient 140. Positive validation may include a confirmation of at least one diagnostic hypothesis 152 that is aligned with, for example, known medical conditions as indicated by at least one matched label 160, reference biomedical signals 172, and any other corroborating patient information encapsulated in EHRs. In other cases, at least one diagnostic hypothesis 152 may be outputted to one or more requesting entities regardless of validation result.

With continued reference to FIG. 1, in one embodiment, outputting at least one diagnostic hypothesis 152 may include retrieving one or more medical literatures in relation to at least one matched label 160, generating, at generative model 112 such as an LLM, a diagnostic response 176 as a function of the one or more retrieved medical literatures, and displaying the generated diagnostic response 176 through a user interface 180 at a display device 184. In some cases, medical literatures may include any medical literatures as described herein. In some cases, retrieving one or more medical literatures may include accessing database 120 or corpus 116 through by querying database 120 or corpus 116 based on at least one matched label 160. As described herein, a “diagnostic response” is a detailed, synthesized output incorporating validated diagnostic hypothesis, supplementary information derived from medical literatures, or both. In one embodiment, diagnostic response 176 may be configured to provide medical professionals with a comprehensive overview of diagnosis, including without limitation, potential causes, implications, associated conditions, suggested next steps for treatment, or further investigation. In some cases, diagnostic response 176 may be tailored to a specific clinical scenario and may be grounded in one or more medical researches and guidelines. In some cases, medical literatures may be organized based on recency, relevance to at least one matched label 160, citation impact, and/or the like. In some cases, retrieving medical literatures may include generating links and/or citations to medical literatures instead of directly obtaining copies. As a non-limiting example, diagnostic response 176 may include one or more inline citations or embedded URLs of one or more related medical literatures. In some cases, diagnostic response 176 may include a synthesized narrative that encompasses key findings in one or more medical literatures that supports at least one diagnostic hypothesis 152.

With continued reference to FIG. 1, in some cases, apparatus 100 may further include a display device 184. As used in this disclosure, a “display device” refers to an electronic device that visually presents information to the entity. In some cases, display device may be configured to project or show visual content generated by computers, video devices, or other electronic mechanisms. In some cases, display devices may include, without limitation, a liquid crystal display (LCD), a cathode ray tube (CRT), a plasma display, a light emitting diode (LED) display, and any combinations thereof. In a non-limiting example, one or more display devices may vary in size, resolution, technology, and functionality. Display device may be able to show any data elements and/or visual elements as listed above in various formats such as, textural, graphical, video among others, in either monochrome or color. In one or more embodiments, transmitting subset of obfuscated data elements 140 may include displaying at least one diagnostic hypothesis 152 and/or diagnostic response 176 at display device using user interface 180. Additionally, or alternatively, processor 104 may be connected to display device 184.

With continued reference to FIG. 1, as used in this disclosure, a “user interface” is a digital display that presents information, options, interactive elements to users in an intuitive and visually appealing manner. In some embodiments, user interface 180 may include at least an interface element. As used in this disclosure, “at least an interface element” is a portion of user interface 180. In a non-limiting example, at least an interface element may include, without limitation, a button, a link, a checkbox, a text entry box and/or window, a drop-down list, a slider, or any other interface element that may occur to a person skilled in the art upon reviewing the entirety of this disclosure. In some embodiments, at least an interface element may include an event handler. An “event handler,” as used in this disclosure, is a module, data structure, function, and/or routine that performs an action on remote device in response to a user interaction with event handler graphic. For instance, and without limitation, an event handler may record data corresponding to user selections of previously populated fields such as drop-down lists and/or text auto-complete and/or default entries, data corresponding to user selections of checkboxes, radio buttons, or the like, potentially along with automatically entered data triggered by such selections, user entry of textual data using a keyboard, touchscreen, speech-to-text program, or the like. Event handler may generate prompts for further information, may compare data to validation rules such as requirements that the data in question be entered within certain numerical ranges, and/or may modify data and/or generate warnings to a user in response to such requirements.

With continued reference to FIG. 1, in some cases, event handler may include a cross-session state variable. As used herein, a “cross-session state variable” is a variable recording obfuscated data elements generated by processor 104 during a previous session. Such data may include, for instance, previously entered text, previous selections of one or more elements as described above, or the like. For instance, and without limitation, cross-session state variable data may represent a request (of at least one diagnostic hypothesis 152) a requesting entity initiated in a past session. Cross-session state variable may be saved using any suitable combination of client-side data storage on remote device and server-side data storage connected to processor 104. In some cases, set of diagnostic hypotheses 124 may be saved wholly or in part as a “cookie” which may include data or an identification of requesting entity to prompt provision of cross-session state variable by processor 104, which may be store in a data store at the requesting entity. In some cases, cross-session state variable may include at least a prior session datum. A “prior session datum” may include any element of data that may be stored in a cross-session state variable. In an embodiment, user interface 180 may be configured to display the at least a prior session datum, for instance and without limitation auto-populating user query data from previous sessions. In a non-limiting example, user interface 180 may include at least one diagnostic hypothesis 152 and/or diagnostic response 176. Advantageously, processor 104 may store previous selections of diagnostic hypothesis such that requesting entity does not have to reselect diagnostic hypothesis each time if there is no substantial change in the biomedical signal.

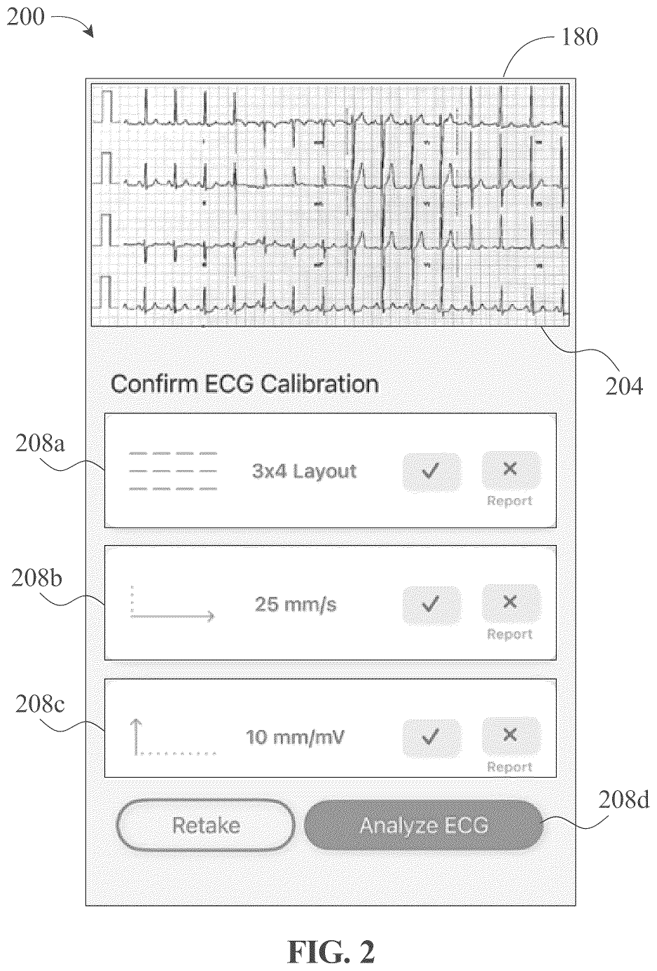

Now referring to FIG. 2, an exemplary embodiment 200 of a user interface 180 is illustrated. In one embodiment, user interface 180 may include at least an interface element, for example, an image box 204, wherein the image box 204 may be configured to display biomedical signal 136 pertaining to patient 140 such as one or more ECGs. As a non-limiting example, biomedical signal 136 may be received from user interface 180 as a user input. User interface 180 may include an ECG preview providing the user with a visual confirmation that one or more ECGs has been recorded and is ready for further processing. Additionally, or alternatively, user interface 180 may include other interface elements having one or more event handlers 208a-c configured to modify image box 204, for example, and without limitation, one or more ECG calibration settings, such as layout option, speed selection, voltage calibration may be displayed, allowing user to confirm the calibration settings for the inputted ECG. In some cases, by default, a “3×4” view option, a speed of 25 mm/s, and a voltage of 10 mm/mv may be set for the given ECG. Once user confirmed ECG calibration settings, processor 104 may proceed to run one or more processing steps as described above upon user interact with event handler 208d configured to submit inputted ECGs.

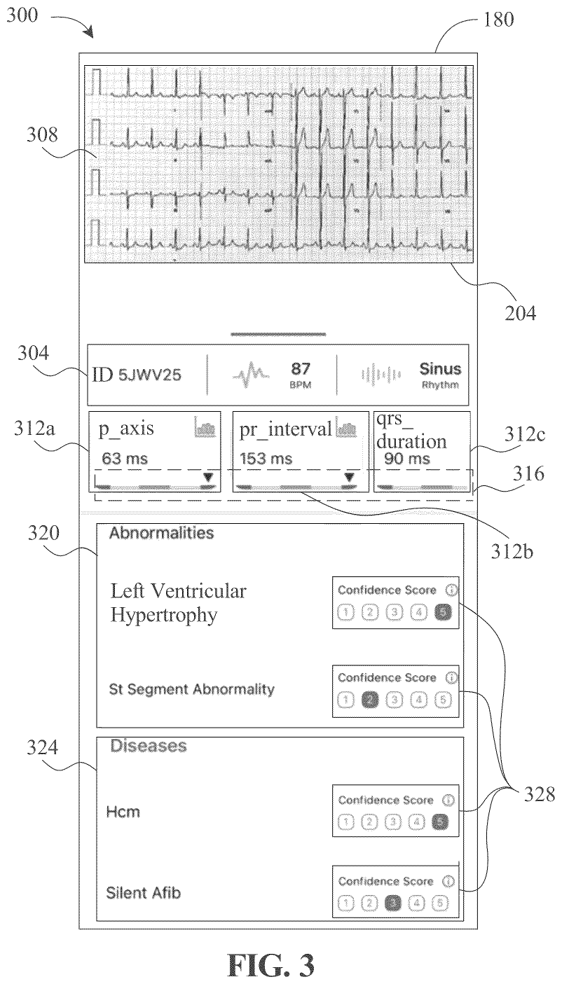

Now referring to FIG. 3, an exemplary embodiment 300 of a user interface 180 is illustrated. User interface 180 may include an interface element containing metadata 304 associated with biomedical signal 136 pertaining to patient 140. As a non-limiting example, data such as patient ID, heart rate (in beats per minute BPM), rhythm type e.g., sinus rhythm may be identified from ECG data 308 and displayed to the user. In some cases, one or more biomedical features such as ECG features 312a-c derived from ECG data 308 may be displayed, for instance, and without limitation, P-axis, PR interval, QRS duration, and/or the like, each with a visual indicator 316 that may denote normal ranges or potential abnormalities. User interface 180 may additionally, or alternatively, include an interface component displaying set of diagnostic hypotheses 124. In one embodiment, set of diagnostic hypotheses 124 may include a list of identified abnormalities 320 such as, without limitation, “left ventricular hypertrophy” and “ST segment abnormality.” In another embodiment, set of diagnostic hypotheses 128 may include a list of identified disease 324 such as, without limitation, “HCM,” and “Silent Afib.” In some cases, each diagnostic hypothesis within set of diagnostic hypotheses 124 may be associated with a confidence level 328 e.g., 1˜5. In some cases, an interface element such as a slider may be implemented, for each confidence level, such that a user may be able to view more details or adjust confidence level based on further review or additional inputs.

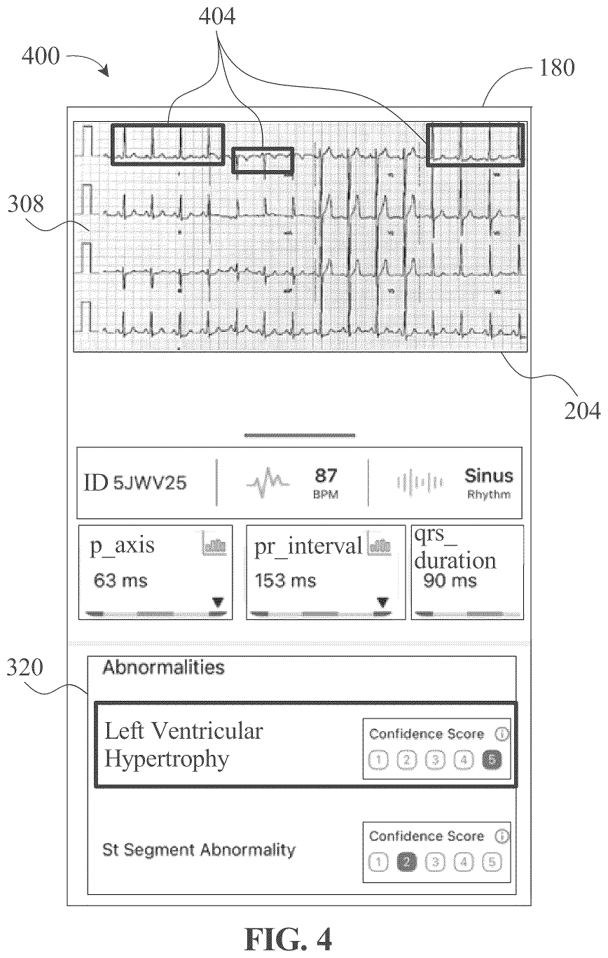

Now referring to FIG. 4, an exemplary embodiment 400 of a user interface 180 is illustrated. In some cases, interface element displaying set of diagnostic hypotheses 124 may be interactive; for instance, and without limitation, user may be able to click any list entry within list of identified abnormalities 320 and/or list of identified disease 324 (not shown). As a non-limiting example, when a user selects an identified abnormality from the list 320, processor 104 may be configured to visually highlight related segments on ECG data 308 that pertain to the selected abnormality. If “left ventricular hypertrophy” is clicked. Processor 104 may highlight the areas on ECG waveforms typically associated with left ventricular hypertrophy, such as high amplitude QRS complexes in certain leads. In one embodiment, user interface 180 may show a color overlay 404 or other graphical element over the waveform to indicate which parts of the ECG contributed to such diagnostic hypothesis. As a non-limiting example, portions of image box 204 representing areas of interest may be modified to thickened color bands shown on the ECG tracing.

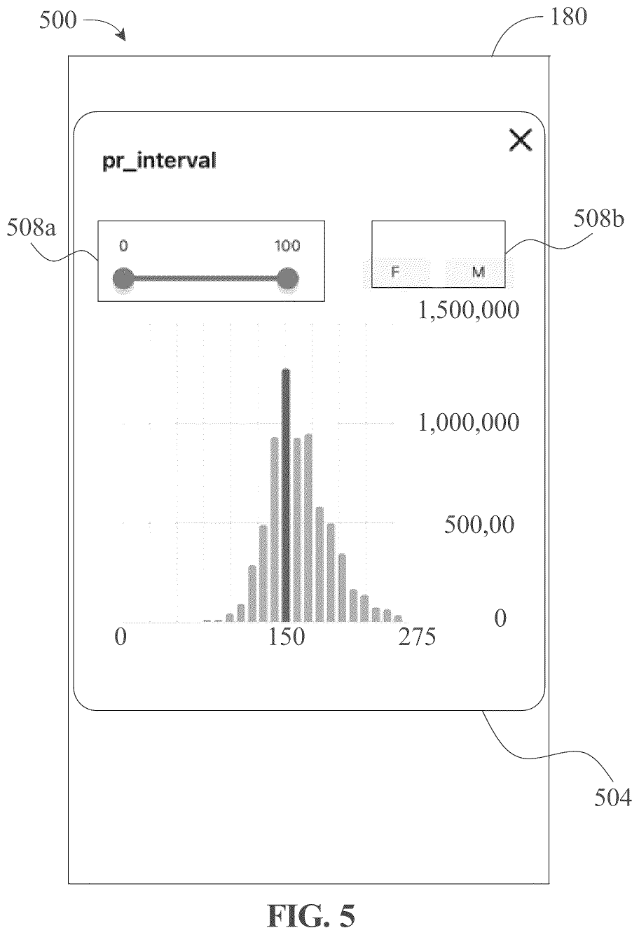

Now referring to FIG. 5, an exemplary embodiment 500 of a user interface 180 is illustrated. Additionally, or alternatively, user interface 180 may be configured to display additional interface element 504 to further visualize one or more identified biomedical features. As a non-limiting example, user may select a particular biomedical feature such as “PR interval,” an additional interface element may be populated, by processor 104, to display a chart, such as a bell curve or histogram showing a distribution of the PR interval values across a population within selected demographic filters. In some cases, one or more event handlers 508a-b may be used to refine displayed data based on, for example, age and biological sex. In some cases, age filtering event handler 508a may include a slider (from 0 to 100 years) while biological sex event handler 508b may include options for “Female (F)” and “Male (M).” Distribution graph may display the distribution of PR interval values within the selected demographic filters with x-axis represents PR interval values in milliseconds, and the y-axis represents the number of patients.

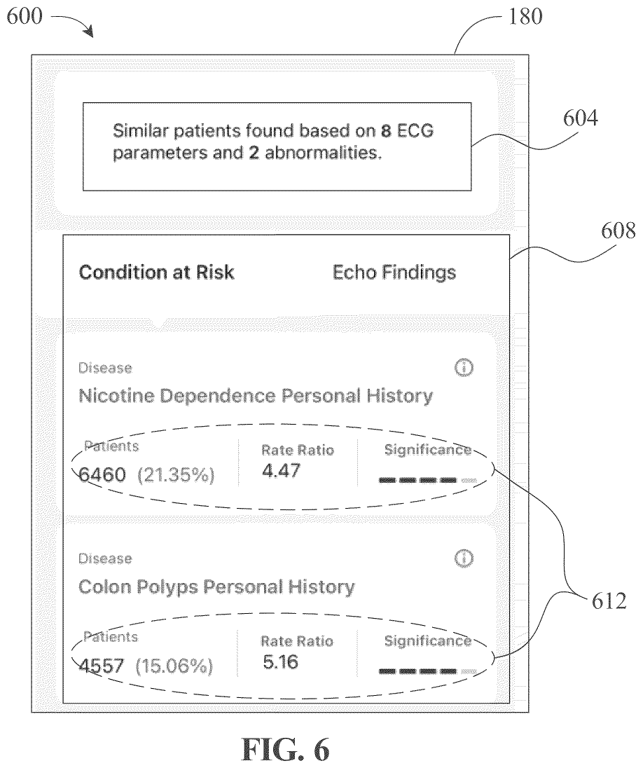

Now referring to FIG. 6, an exemplary embodiment 600 of a user interface 180 is illustrated. User interface 180 may include an interface element displaying diagnostic response 176. In one embodiment, diagnostic response 176 may include validation results such as, without limitation, validation summary 604, list of conditions or risk factors 608 associated with patient's 140 ECG findings, statistical data 612, and/or the like generated based on selected diagnostic hypotheses. As a non-limiting example, validation summary 604 may be presented as an information banner stating, “similar patients found based on 8 ECG parameters and 2 abnormalities.” List of conditions or risk factors 608 may include, without limitation, information regarding patient's 140 nicotine dependence personal history, colon polyps personal history, history of falling unspecified history, and/or any other information that may not be directly related to the heart but could represent comorbidities or risk factors identified in similar patient populations. In some cases, each listed condition or risk factor within the list 608 may be associated with statistical data 612 such as, without limitation, patient number and percentage, rate ratio, significance indicator representing statistical significance of the association between the ECG finding and the corresponding listed condition. As a non-limiting example, list of conditions or risk factors 608 and associated statistical data 612 may be results of querying medical repository 164 as described above.

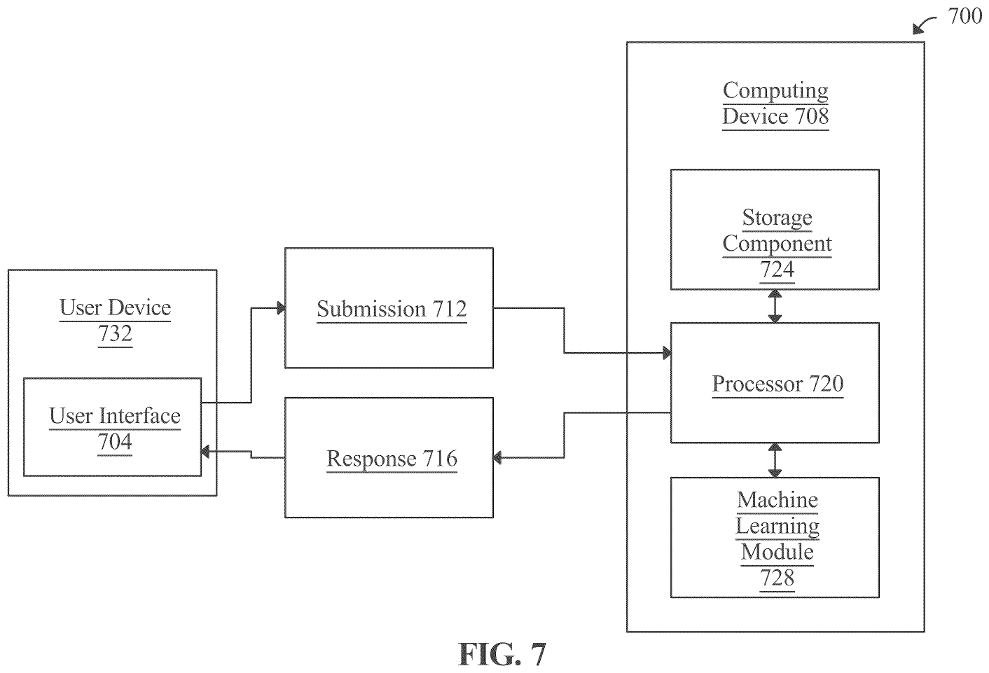

Now referring to FIG. 7, an exemplary embodiment of a chatbot 700 is illustrated. In one embodiment, generative model 112 such as an LLM may include a dialog agent; for instance, a chatbot 700. In some embodiments, user may communicate with apparatus 100 using chatbot 700. Similarly, apparatus 100 may deliver output to the user using chatbot 700. According to some embodiments, user interface 704 on user device 732 may be communicative with a computing device 708 that is configured to operate a chatbot. In some embodiments, user interface 704 may be local to user device 732. In some embodiments, user interface 704 may be local to computing device 708. Alternatively, or additionally, in some cases, user interface 704 may remote to user device 732 and communicative with user device 732, by way of one or more networks, such as without limitation the internet. Alternatively, or additionally, one or more user interfaces may communicate with computing device 708 using telephonic devices and networks, such as without limitation fax machines, short message service (SMS), or multimedia message service (MMS). Commonly, user communicate with computing device 708 using text-based communication, for example without limitation using a character encoding protocol, such as American Standard for Information Interchange (ASCII). Typically, user interfaces conversationally interface with a chatbot, by way of at least a submission, from a user interface to the chatbot, and a response, from the chatbot to the user interface. For example, user interface 704 may interface with a chatbot using submission 712 and response 716. In some embodiments, submission 712 and/or response 716 may use text-based communication. In some embodiments, submission 712 and/or response 716 may use audio communication. As a non-limiting example, user may submit a submission 712 including a prompt “what is appropriate treatment for a patient with X condition and X disease,” and chatbot 700 may provide a response 716 containing one or more relevant medical literatures or published guidelines retrieved from database 120 as described above.

Still referring to FIG. 7, submission 712, once received by computing device 708 operating a chatbot, may be processed by a processor 720. In some embodiments, processor 720 processes submission 712 using one or more of keyword recognition, pattern matching, and natural language processing. In some embodiments, processor employs real-time learning with evolutionary algorithms. In some cases, processor 720 may retrieve a pre-prepared response from at least a storage component 724, based upon submission 712. Alternatively, or additionally, in some embodiments, processor 720 communicates a response 716 without first receiving a submission, thereby initiating conversation. In some cases, processor 720 communicates an inquiry to user interface 704; and processor 720 is configured to process an answer to the inquiry in a following submission from the user interface. In some cases, an answer to an inquiry present within a submission from a user device may be used by computing device 708 as an input to another function. In some embodiments, computing device 708 may include machine learning module 728. Machine learning module 728 may include any machine learning models described herein, such as, without limitation, one or more LLMs. In some embodiments, submission 712 may be input into a trained machine learning model within machine learning module 728. In some embodiments, submission 712 may undergo one or more processing steps before being input into a machine learning model. In some embodiments, submission 712 may be used to train a machine learning model within machine learning module 728.

Referring now to FIG. 8, an exemplary embodiment of a machine-learning module 800 that may perform one or more machine-learning processes as described in this disclosure is illustrated. Machine-learning module may perform determinations, classification, and/or analysis steps, methods, processes, or the like as described in this disclosure using machine learning processes. A “machine learning process,” as used in this disclosure, is a process that automatedly uses training data 804 to generate an algorithm instantiated in hardware or software logic, data structures, and/or functions that will be performed by a computing device/module to produce outputs 808 given data provided as inputs 812; this is in contrast to a non-machine learning software program where the commands to be executed are determined in advance by a user and written in a programming language.

Still referring to FIG. 8, “training data,” as used herein, is data containing correlations that a machine-learning process may use to model relationships between two or more categories of data elements. For instance, and without limitation, training data 804 may include a plurality of data entries, also known as “training examples,” each entry representing a set of data elements that were recorded, received, and/or generated together; data elements may be correlated by shared existence in a given data entry, by proximity in a given data entry, or the like. Multiple data entries in training data 804 may evince one or more trends in correlations between categories of data elements; for instance, and without limitation, a higher value of a first data element belonging to a first category of data element may tend to correlate to a higher value of a second data element belonging to a second category of data element, indicating a possible proportional or other mathematical relationship linking values belonging to the two categories. Multiple categories of data elements may be related in training data 804 according to various correlations; correlations may indicate causative and/or predictive links between categories of data elements, which may be modeled as relationships such as mathematical relationships by machine-learning processes as described in further detail below. Training data 804 may be formatted and/or organized by categories of data elements, for instance by associating data elements with one or more descriptors corresponding to categories of data elements. As a non-limiting example, training data 804 may include data entered in standardized forms by persons or processes, such that entry of a given data element in a given field in a form may be mapped to one or more descriptors of categories. Elements in training data 804 may be linked to descriptors of categories by tags, tokens, or other data elements; for instance, and without limitation, training data 804 may be provided in fixed-length formats, formats linking positions of data to categories such as comma-separated value (CSV) formats and/or self-describing formats such as extensible markup language (XML), JavaScript Object Notation (JSON), or the like, enabling processes or devices to detect categories of data.

Alternatively, or additionally, and continuing to refer to FIG. 8, training data 804 may include one or more elements that are not categorized; that is, training data 804 may not be formatted or contain descriptors for some elements of data. Machine-learning algorithms and/or other processes may sort training data 804 according to one or more categorizations using, for instance, natural language processing algorithms, tokenization, detection of correlated values in raw data and the like; categories may be generated using correlation and/or other processing algorithms. As a non-limiting example, in a corpus of text, phrases making up a number “n” of compound words, such as nouns modified by other nouns, may be identified according to a statistically significant prevalence of n-grams containing such words in a particular order; such an n-gram may be categorized as an element of language such as a “word” to be tracked similarly to single words, generating a new category as a result of statistical analysis. Similarly, in a data entry including some textual data, a person's name may be identified by reference to a list, dictionary, or other compendium of terms, permitting ad-hoc categorization by machine-learning algorithms, and/or automated association of data in the data entry with descriptors or into a given format. The ability to categorize data entries automatedly may enable the same training data 804 to be made applicable for two or more distinct machine-learning algorithms as described in further detail below. Training data 804 used by machine-learning module 800 may correlate any input data as described in this disclosure to any output data as described in this disclosure. Exemplary training data 804 may include a plurality of diagnostic hypotheses as input correlated to a plurality of labels as output.

Further referring to FIG. 8, training data may be filtered, sorted, and/or selected using one or more supervised and/or unsupervised machine-learning processes and/or models as described in further detail below; such models may include without limitation a training data classifier 816. Training data classifier 816 may include a “classifier,” which as used in this disclosure is a machine-learning model as defined below, such as a data structure representing and/or using a mathematical model, neural net, or program generated by a machine learning algorithm known as a “classification algorithm,” as described in further detail below, that sorts inputs into categories or bins of data, outputting the categories or bins of data and/or labels associated therewith. A classifier may be configured to output at least a datum that labels or otherwise identifies a set of data that are clustered together, found to be close under a distance metric as described below, or the like. A distance metric may include any norm, such as, without limitation, a Pythagorean norm. Machine-learning module 800 may generate a classifier using a classification algorithm, defined as a processes whereby a computing device and/or any module and/or component operating thereon derives a classifier from training data 804. Classification may be performed using, without limitation, linear classifiers such as without limitation logistic regression and/or naive Bayes classifiers, nearest neighbor classifiers such as k-nearest neighbors classifiers, support vector machines, least squares support vector machines, fisher's linear discriminant, quadratic classifiers, decision trees, boosted trees, random forest classifiers, learning vector quantization, and/or neural network-based classifiers.

Still referring to FIG. 8, Computing device may be configured to generate a classifier using a Naïve Bayes classification algorithm. Naïve Bayes classification algorithm generates classifiers by assigning class labels to problem instances, represented as vectors of element values. Class labels are drawn from a finite set. Naïve Bayes classification algorithm may include generating a family of algorithms that assume that the value of a particular element is independent of the value of any other element, given a class variable. Naïve Bayes classification algorithm may be based on Bayes Theorem expressed as P(A/B)=P(B/A) P(A)÷P(B), where P(A/B) is the probability of hypothesis A given data B also known as posterior probability; P(B/A) is the probability of data B given that the hypothesis A was true; P(A) is the probability of hypothesis A being true regardless of data also known as prior probability of A; and P(B) is the probability of the data regardless of the hypothesis. A naïve Bayes algorithm may be generated by first transforming training data into a frequency table. Computing device may then calculate a likelihood table by calculating probabilities of different data entries and classification labels. Computing device may utilize a naïve Bayes equation to calculate a posterior probability for each class. A class containing the highest posterior probability is the outcome of prediction. Naïve Bayes classification algorithm may include a gaussian model that follows a normal distribution. Naïve Bayes classification algorithm may include a multinomial model that is used for discrete counts. Naïve Bayes classification algorithm may include a Bernoulli model that may be utilized when vectors are binary.

With continued reference to FIG. 8, Computing device may be configured to generate a classifier using a K-nearest neighbors (KNN) algorithm. A “K-nearest neighbors algorithm” as used in this disclosure, includes a classification method that utilizes feature similarity to analyze how closely out-of-sample-features resemble training data to classify input data to one or more clusters and/or categories of features as represented in training data; this may be performed by representing both training data and input data in vector forms, and using one or more measures of vector similarity to identify classifications within training data, and to determine a classification of input data. K-nearest neighbors algorithm may include specifying a K-value, or a number directing the classifier to select the k most similar entries training data to a given sample, determining the most common classifier of the entries in the database, and classifying the known sample; this may be performed recursively and/or iteratively to generate a classifier that may be used to classify input data as further samples. For instance, an initial set of samples may be performed to cover an initial heuristic and/or “first guess” at an output and/or relationship, which may be seeded, without limitation, using expert input received according to any process as described herein. As a non-limiting example, an initial heuristic may include a ranking of associations between inputs and elements of training data. Heuristic may include selecting some number of highest-ranking associations and/or training data elements.

With continued reference to FIG. 8, generating k-nearest neighbors algorithm may generate a first vector output containing a data entry cluster, generating a second vector output containing an input data, and calculate the distance between the first vector output and the second vector output using any suitable norm such as cosine similarity, Euclidean distance measurement, or the like. Each vector output may be represented, without limitation, as an n-tuple of values, where n is at least two values. Each value of n-tuple of values may represent a measurement or other quantitative value associated with a given category of data, or attribute, examples of which are provided in further detail below; a vector may be represented, without limitation, in n-dimensional space using an axis per category of value represented in n-tuple of values, such that a vector has a geometric direction characterizing the relative quantities of attributes in the n-tuple as compared to each other. Two vectors may be considered equivalent where their directions, and/or the relative quantities of values within each vector as compared to each other, are the same; thus, as a non-limiting example, a vector represented as [5, 10, 15] may be treated as equivalent, for purposes of this disclosure, as a vector represented as [1, 2, 3]. Vectors may be more similar where their directions are more similar, and more different where their directions are more divergent; however, vector similarity may alternatively or additionally be determined using averages of similarities between like attributes, or any other measure of similarity suitable for any n-tuple of values, or aggregation of numerical similarity measures for the purposes of loss functions as described in further detail below. Any vectors as described herein may be scaled, such that each vector represents each attribute along an equivalent scale of values. Each vector may be “normalized,” or divided by a “length” attribute, such as a length attribute l as derived using a Pythagorean norm:

$$l = \sqrt{{\sum }_{i = 0}^{n} {{a}_{i}}^{2}} ,$$

where aᵢ is attribute number i of the vector. Scaling and/or normalization may function to make vector comparison independent of absolute quantities of attributes, while preserving any dependency on similarity of attributes; this may, for instance, be advantageous where cases represented in training data are represented by different quantities of samples, which may result in proportionally equivalent vectors with divergent values.

With further reference to FIG. 8, training examples for use as training data may be selected from a population of potential examples according to cohorts relevant to an analytical problem to be solved, a classification task, or the like. Alternatively, or additionally, training data may be selected to span a set of likely circumstances or inputs for a machine-learning model and/or process to encounter when deployed. For instance, and without limitation, for each category of input data to a machine-learning process or model that may exist in a range of values in a population of phenomena such as images, user data, process data, physical data, or the like, a computing device, processor, and/or machine-learning model may select training examples representing each possible value on such a range and/or a representative sample of values on such a range. Selection of a representative sample may include selection of training examples in proportions matching a statistically determined and/or predicted distribution of such values according to relative frequency, such that, for instance, values encountered more frequently in a population of data so analyzed are represented by more training examples than values that are encountered less frequently. Alternatively, or additionally, a set of training examples may be compared to a collection of representative values in a database and/or presented to a user, so that a process can detect, automatically or via user input, one or more values that are not included in the set of training examples. Computing device, processor, and/or module may automatically generate a missing training example; this may be done by receiving and/or retrieving a missing input and/or output value and correlating the missing input and/or output value with a corresponding output and/or input value collocated in a data record with the retrieved value, provided by a user and/or other device, or the like.

Continuing to refer to FIG. 8, computer, processor, and/or module may be configured to preprocess training data. “Preprocessing” training data, as used in this disclosure, is transforming training data from raw form to a format that can be used for training a machine learning model. Preprocessing may include sanitizing, feature selection, feature scaling, data augmentation and the like.

Still referring to FIG. 8, computer, processor, and/or module may be configured to sanitize training data. “Sanitizing” training data, as used in this disclosure, is a process whereby training examples are removed that interfere with convergence of a machine-learning model and/or process to a useful result. For instance, and without limitation, a training example may include an input and/or output value that is an outlier from typically encountered values, such that a machine-learning algorithm using the training example will be adapted to an unlikely amount as an input and/or output; a value that is more than a threshold number of standard deviations away from an average, mean, or expected value, for instance, may be eliminated. Alternatively, or additionally, one or more training examples may be identified as having poor quality data, where “poor quality” is defined as having a signal to noise ratio below a threshold value. Sanitizing may include steps such as removing duplicative or otherwise redundant data, interpolating missing data, correcting data errors, standardizing data, identifying outliers, and the like. In a nonlimiting example, sanitization may include utilizing algorithms for identifying duplicate entries or spell-check algorithms.

As a non-limiting example, and with further reference to FIG. 8, images used to train an image classifier or other machine-learning model and/or process that takes images as inputs or generates images as outputs may be rejected if image quality is below a threshold value. For instance, and without limitation, computing device, processor, and/or module may perform blur detection, and eliminate one or more Blur detection may be performed, as a non-limiting example, by taking Fourier transform, or an approximation such as a Fast Fourier Transform (FFT) of the image and analyzing a distribution of low and high frequencies in the resulting frequency-domain depiction of the image; numbers of high-frequency values below a threshold level may indicate blurriness. As a further non-limiting example, detection of blurriness may be performed by convolving an image, a channel of an image, or the like with a Laplacian kernel; this may generate a numerical score reflecting a number of rapid changes in intensity shown in the image, such that a high score indicates clarity, and a low score indicates blurriness. Blurriness detection may be performed using a gradient-based operator, which measures operators based on the gradient or first derivative of an image, based on the hypothesis that rapid changes indicate sharp edges in the image, and thus are indicative of a lower degree of blurriness. Blur detection may be performed using Wavelet-based operator, which takes advantage of the capability of coefficients of the discrete wavelet transform to describe the frequency and spatial content of images. Blur detection may be performed using statistics-based operators take advantage of several image statistics as texture descriptors in order to compute a focus level. Blur detection may be performed by using discrete cosine transform (DCT) coefficients in order to compute a focus level of an image from its frequency content.

Continuing to refer to FIG. 8, computing device, processor, and/or module may be configured to precondition one or more training examples. For instance, and without limitation, where a machine learning model and/or process has one or more inputs and/or outputs requiring, transmitting, or receiving a certain number of bits, samples, or other units of data, one or more training examples' elements to be used as or compared to inputs and/or outputs may be modified to have such a number of units of data. For instance, a computing device, processor, and/or module may convert a smaller number of units, such as in a low pixel count image, into a desired number of units, for instance by upsampling and interpolating. As a non-limiting example, a low pixel count image may have 100 pixels, however a desired number of pixels may be 128. Processor may interpolate the low pixel count image to convert the 100 pixels into 128 pixels. It should also be noted that one of ordinary skill in the art, upon reading this disclosure, would know the various methods to interpolate a smaller number of data units such as samples, pixels, bits, or the like to a desired number of such units. In some instances, a set of interpolation rules may be trained by sets of highly detailed inputs and/or outputs and corresponding inputs and/or outputs downsampled to smaller numbers of units, and a neural network or other machine learning model that is trained to predict interpolated pixel values using the training data. As a non-limiting example, a sample input and/or output, such as a sample picture, with sample-expanded data units (e.g., pixels added between the original pixels) may be input to a neural network or machine-learning model and output a pseudo replica sample-picture with dummy values assigned to pixels between the original pixels based on a set of interpolation rules. As a non-limiting example, in the context of an image classifier, a machine-learning model may have a set of interpolation rules trained by sets of highly detailed images and images that have been downsampled to smaller numbers of pixels, and a neural network or other machine learning model that is trained using those examples to predict interpolated pixel values in a facial picture context. As a result, an input with sample-expanded data units (the ones added between the original data units, with dummy values) may be run through a trained neural network and/or model, which may fill in values to replace the dummy values. Alternatively, or additionally, processor, computing device, and/or module may utilize sample expander methods, a low-pass filter, or both. As used in this disclosure, a “low-pass filter” is a filter that passes signals with a frequency lower than a selected cutoff frequency and attenuates signals with frequencies higher than the cutoff frequency. The exact frequency response of the filter depends on the filter design. Computing device, processor, and/or module may use averaging, such as luma or chroma averaging in images, to fill in data units in between original data units.

In some embodiments, and with continued reference to FIG. 8, computing device, processor, and/or module may down-sample elements of a training example to a desired lower number of data elements. As a non-limiting example, a high pixel count image may have 256 pixels, however a desired number of pixels may be 128. Processor may down-sample the high pixel count image to convert the 256 pixels into 128 pixels. In some embodiments, processor may be configured to perform downsampling on data. Downsampling, also known as decimation, may include removing every Nth entry in a sequence of samples, all but every Nth entry, or the like, which is a process known as “compression,” and may be performed, for instance by an N-sample compressor implemented using hardware or software. Anti-aliasing and/or anti-imaging filters, and/or low-pass filters, may be used to clean up side-effects of compression.

Further referring to FIG. 8, feature selection includes narrowing and/or filtering training data to exclude features and/or elements, or training data including such elements, that are not relevant to a purpose for which a trained machine-learning model and/or algorithm is being trained, and/or collection of features and/or elements, or training data including such elements, on the basis of relevance or utility for an intended task or purpose for a trained machine-learning model and/or algorithm is being trained. Feature selection may be implemented, without limitation, using any process described in this disclosure, including without limitation using training data classifiers, exclusion of outliers, or the like.

With continued reference to FIG. 8, feature scaling may include, without limitation, normalization of data entries, which may be accomplished by dividing numerical fields by norms thereof, for instance as performed for vector normalization. Feature scaling may include absolute maximum scaling, wherein each quantitative datum is divided by the maximum absolute value of all quantitative data of a set or subset of quantitative data. Feature scaling may include min-max scaling, in which each value X has a minimum value Xₘᵢₙ in a set or subset of values subtracted therefrom, with the result divided by the range of the values, give maximum value in the set or subset Xₘₐₓ:

$${X}_{new} = \frac{X - {X}_{min}}{{X}_{max} - {X}_{min}} .$$

Feature scaling may include mean normalization, which involves use of a mean value of a set and/or subset of values, Xₘₑₐₙ with maximum and minimum values

$${X}_{new} = \frac{X - {X}_{mean}}{{X}_{max} - {X}_{min}} .$$

Feature scaling may include standardization, where a difference between X and Xₘₑₐₙ is divided by a standard deviation σ of a set or subset of values:

$${X}_{new} = \frac{X - {X}_{mean}}{σ} .$$

Scaling may be performed using a median value of a set or subset X_{median} and/or interquartile range (IQR), which represents the difference between the 25^{th} percentile value and the 50^{th} percentile value (or closest values thereto by a rounding protocol), such as:

$${X}_{new} = \frac{X - {X}_{median}}{IQR} .$$

Persons skilled in the art, upon reviewing the entirety of this disclosure, will be aware of various alternative or additional approaches that may be used for feature scaling.

Further referring to FIG. 8, computing device, processor, and/or module may be configured to perform one or more processes of data augmentation. “Data augmentation” as used in this disclosure is addition of data to a training set using elements and/or entries already in the dataset. Data augmentation may be accomplished, without limitation, using interpolation, generation of modified copies of existing entries and/or examples, and/or one or more generative AI processes, for instance using deep neural networks and/or generative adversarial networks; generative processes may be referred to alternatively in this context as “data synthesis” and as creating “synthetic data.” Augmentation may include performing one or more transformations on data, such as geometric, color space, affine, brightness, cropping, and/or contrast transformations of images.

Still referring to FIG. 8, machine-learning module 800 may be configured to perform a lazy-learning process 820 and/or protocol, which may alternatively be referred to as a “lazy loading” or “call-when-needed” process and/or protocol, may be a process whereby machine learning is conducted upon receipt of an input to be converted to an output, by combining the input and training set to derive the algorithm to be used to produce the output on demand. For instance, an initial set of simulations may be performed to cover an initial heuristic and/or “first guess” at an output and/or relationship. As a non-limiting example, an initial heuristic may include a ranking of associations between inputs and elements of training data 804. Heuristic may include selecting some number of highest-ranking associations and/or training data 804 elements. Lazy learning may implement any suitable lazy learning algorithm, including without limitation a K-nearest neighbors algorithm, a lazy naïve Bayes algorithm, or the like; persons skilled in the art, upon reviewing the entirety of this disclosure, will be aware of various lazy-learning algorithms that may be applied to generate outputs as described in this disclosure, including without limitation lazy learning applications of machine-learning algorithms as described in further detail below.

Alternatively, or additionally, and with continued reference to FIG. 8, machine-learning processes as described in this disclosure may be used to generate machine-learning models 824. A “machine-learning model,” as used in this disclosure, is a data structure representing and/or instantiating a mathematical and/or algorithmic representation of a relationship between inputs and outputs, as generated using any machine-learning process including without limitation any process as described above, and stored in memory; an input is submitted to a machine-learning model 824 once created, which generates an output based on the relationship that was derived. For instance, and without limitation, a linear regression model, generated using a linear regression algorithm, may compute a linear combination of input data using coefficients derived during machine-learning processes to calculate an output datum. As a further non-limiting example, a machine-learning model 824 may be generated by creating an artificial neural network, such as a convolutional neural network comprising an input layer of nodes, one or more intermediate layers, and an output layer of nodes. Connections between nodes may be created via the process of “training” the network, in which elements from a training data 804 set are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning.

Still referring to FIG. 8, machine-learning algorithms may include at least a supervised machine-learning process 828. At least a supervised machine-learning process 828, as defined herein, include algorithms that receive a training set relating a number of inputs to a number of outputs, and seek to generate one or more data structures representing and/or instantiating one or more mathematical relations relating inputs to outputs, where each of the one or more mathematical relations is optimal according to some criterion specified to the algorithm using some scoring function. For instance, a supervised learning algorithm may a plurality of diagnostic hypotheses as described above as inputs, a plurality of labels as outputs, and a scoring function representing a desired form of relationship to be detected between inputs and outputs; scoring function may, for instance, seek to maximize the probability that a given input and/or combination of elements inputs is associated with a given output to minimize the probability that a given input is not associated with a given output. Scoring function may be expressed as a risk function representing an “expected loss” of an algorithm relating inputs to outputs, where loss is computed as an error function representing a degree to which a prediction generated by the relation is incorrect when compared to a given input-output pair provided in training data 804. Persons skilled in the art, upon reviewing the entirety of this disclosure, will be aware of various possible variations of at least a supervised machine-learning process 828 that may be used to determine relation between inputs and outputs. Supervised machine-learning processes may include classification algorithms as defined above.

With further reference to FIG. 8, training a supervised machine-learning process may include, without limitation, iteratively updating coefficients, biases, weights based on an error function, expected loss, and/or risk function. For instance, an output generated by a supervised machine-learning model using an input example in a training example may be compared to an output example from the training example; an error function may be generated based on the comparison, which may include any error function suitable for use with any machine-learning algorithm described in this disclosure, including a square of a difference between one or more sets of compared values or the like. Such an error function may be used in turn to update one or more weights, biases, coefficients, or other parameters of a machine-learning model through any suitable process including without limitation gradient descent processes, least-squares processes, and/or other processes described in this disclosure. This may be done iteratively and/or recursively to gradually tune such weights, biases, coefficients, or other parameters. Updating may be performed, in neural networks, using one or more back-propagation algorithms. Iterative and/or recursive updates to weights, biases, coefficients, or other parameters as described above may be performed until currently available training data is exhausted and/or until a convergence test is passed, where a “convergence test” is a test for a condition selected as indicating that a model and/or weights, biases, coefficients, or other parameters thereof has reached a degree of accuracy. A convergence test may, for instance, compare a difference between two or more successive errors or error function values, where differences below a threshold amount may be taken to indicate convergence. Alternatively, or additionally, one or more errors and/or error function values evaluated in training iterations may be compared to a threshold.

Still referring to FIG. 8, a computing device, processor, and/or module may be configured to perform method, method step, sequence of method steps and/or algorithm described in reference to this figure, in any order and with any degree of repetition. For instance, a computing device, processor, and/or module may be configured to perform a single step, sequence and/or algorithm repeatedly until a desired or commanded outcome is achieved; repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks. A computing device, processor, and/or module may perform any step, sequence of steps, or algorithm in parallel, such as simultaneously and/or substantially simultaneously performing a step two or more times using two or more parallel threads, processor cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

Further referring to FIG. 8, machine learning processes may include at least an unsupervised machine-learning processes 832. An unsupervised machine-learning process, as used herein, is a process that derives inferences in datasets without regard to labels; as a result, an unsupervised machine-learning process may be free to discover any structure, relationship, and/or correlation provided in the data. Unsupervised processes 832 may not require a response variable; unsupervised processes 832 may be used to find interesting patterns and/or inferences between variables, to determine a degree of correlation between two or more variables, or the like.

Still referring to FIG. 8, machine-learning module 800 may be designed and configured to create a machine-learning model 824 using techniques for development of linear regression models. Linear regression models may include ordinary least squares regression, which aims to minimize the square of the difference between predicted outcomes and actual outcomes according to an appropriate norm for measuring such a difference (e.g., a vector-space distance norm); coefficients of the resulting linear equation may be modified to improve minimization. Linear regression models may include ridge regression methods, where the function to be minimized includes the least-squares function plus term multiplying the square of each coefficient by a scalar amount to penalize large coefficients. Linear regression models may include least absolute shrinkage and selection operator (LASSO) models, in which ridge regression is combined with multiplying the least-squares term by a factor of 1 divided by double the number of samples. Linear regression models may include a multi-task lasso model wherein the norm applied in the least-squares term of the lasso model is the Frobenius norm amounting to the square root of the sum of squares of all terms. Linear regression models may include the elastic net model, a multi-task elastic net model, a least angle regression model, a LARS lasso model, an orthogonal matching pursuit model, a Bayesian regression model, a logistic regression model, a stochastic gradient descent model, a perceptron model, a passive aggressive algorithm, a robustness regression model, a Huber regression model, or any other suitable model that may occur to persons skilled in the art upon reviewing the entirety of this disclosure. Linear regression models may be generalized in an embodiment to polynomial regression models, whereby a polynomial equation (e.g., a quadratic, cubic or higher-order equation) providing a best predicted output/actual output fit is sought; similar methods to those described above may be applied to minimize error functions, as will be apparent to persons skilled in the art upon reviewing the entirety of this disclosure.

Continuing to refer to FIG. 8, machine-learning algorithms may include, without limitation, linear discriminant analysis. Machine-learning algorithm may include quadratic discriminant analysis. Machine-learning algorithms may include kernel ridge regression. Machine-learning algorithms may include support vector machines, including without limitation support vector classification-based regression processes. Machine-learning algorithms may include stochastic gradient descent algorithms, including classification and regression algorithms based on stochastic gradient descent. Machine-learning algorithms may include nearest neighbors algorithms. Machine-learning algorithms may include various forms of latent space regularization such as variational regularization. Machine-learning algorithms may include Gaussian processes such as Gaussian Process Regression. Machine-learning algorithms may include cross-decomposition algorithms, including partial least squares and/or canonical correlation analysis. Machine-learning algorithms may include naïve Bayes methods. Machine-learning algorithms may include algorithms based on decision trees, such as decision tree classification or regression algorithms. Machine-learning algorithms may include ensemble methods such as bagging meta-estimator, forest of randomized trees, AdaBoost, gradient tree boosting, and/or voting classifier methods. Machine-learning algorithms may include neural net algorithms, including convolutional neural net processes.

Still referring to FIG. 8, a machine-learning model and/or process may be deployed or instantiated by incorporation into a program, apparatus, system and/or module. For instance, and without limitation, a machine-learning model, neural network, and/or some or all parameters thereof may be stored and/or deployed in any memory or circuitry. Parameters such as coefficients, weights, and/or biases may be stored as circuit-based constants, such as arrays of wires and/or binary inputs and/or outputs set at logic “1” and “0” voltage levels in a logic circuit to represent a number according to any suitable encoding system including twos complement or the like or may be stored in any volatile and/or non-volatile memory. Similarly, mathematical operations and input and/or output of data to or from models, neural network layers, or the like may be instantiated in hardware circuitry and/or in the form of instructions in firmware, machine-code such as binary operation code instructions, assembly language, or any higher-order programming language. Any technology for hardware and/or software instantiation of memory, instructions, data structures, and/or algorithms may be used to instantiate a machine-learning process and/or model, including without limitation any combination of production and/or configuration of non-reconfigurable hardware elements, circuits, and/or modules such as without limitation ASICs, production and/or configuration of reconfigurable hardware elements, circuits, and/or modules such as without limitation FPGAs, production and/or of non-reconfigurable and/or configuration non-rewritable memory elements, circuits, and/or modules such as without limitation non-rewritable ROM, production and/or configuration of reconfigurable and/or rewritable memory elements, circuits, and/or modules such as without limitation rewritable ROM or other memory technology described in this disclosure, and/or production and/or configuration of any computing device and/or component thereof as described in this disclosure. Such deployed and/or instantiated machine-learning model and/or algorithm may receive inputs from any other process, module, and/or component described in this disclosure, and produce outputs to any other process, module, and/or component described in this disclosure.

Continuing to refer to FIG. 8, any process of training, retraining, deployment, and/or instantiation of any machine-learning model and/or algorithm may be performed and/or repeated after an initial deployment and/or instantiation to correct, refine, and/or improve the machine-learning model and/or algorithm. Such retraining, deployment, and/or instantiation may be performed as a periodic or regular process, such as retraining, deployment, and/or instantiation at regular elapsed time periods, after some measure of volume such as a number of bytes or other measures of data processed, a number of uses or performances of processes described in this disclosure, or the like, and/or according to a software, firmware, or other update schedule. Alternatively or additionally, retraining, deployment, and/or instantiation may be event-based, and may be triggered, without limitation, by user inputs indicating sub-optimal or otherwise problematic performance and/or by automated field testing and/or auditing processes, which may compare outputs of machine-learning models and/or algorithms, and/or errors and/or error functions thereof, to any thresholds, convergence tests, or the like, and/or may compare outputs of processes described herein to similar thresholds, convergence tests or the like. Event-based retraining, deployment, and/or instantiation may alternatively or additionally be triggered by receipt and/or generation of one or more new training examples; a number of new training examples may be compared to a preconfigured threshold, where exceeding the preconfigured threshold may trigger retraining, deployment, and/or instantiation.

Still referring to FIG. 8, retraining and/or additional training may be performed using any process for training described above, using any currently or previously deployed version of a machine-learning model and/or algorithm as a starting point. Training data for retraining may be collected, preconditioned, sorted, classified, sanitized, or otherwise processed according to any process described in this disclosure. Training data may include, without limitation, training examples including inputs and correlated outputs used, received, and/or generated from any version of any system, module, machine-learning model or algorithm, apparatus, and/or method described in this disclosure; such examples may be modified and/or labeled according to user feedback or other processes to indicate desired results, and/or may have actual or measured results from a process being modeled and/or predicted by system, module, machine-learning model or algorithm, apparatus, and/or method as “desired” results to be compared to outputs for training processes as described above.

Redeployment may be performed using any reconfiguring and/or rewriting of reconfigurable and/or rewritable circuit and/or memory elements; alternatively, redeployment may be performed by production of new hardware and/or software components, circuits, instructions, or the like, which may be added to and/or may replace existing hardware and/or software components, circuits, instructions, or the like.

Further referring to FIG. 8, one or more processes or algorithms described above may be performed by at least a dedicated hardware unit 836. A “dedicated hardware unit,” for the purposes of this figure, is a hardware component, circuit, or the like, aside from a principal control circuit and/or processor performing method steps as described in this disclosure, that is specifically designated or selected to perform one or more specific tasks and/or processes described in reference to this figure, such as without limitation preconditioning and/or sanitization of training data and/or training a machine-learning algorithm and/or model. A dedicated hardware unit 836 may include, without limitation, a hardware unit that can perform iterative or massed calculations, such as matrix-based calculations to update or tune parameters, weights, coefficients, and/or biases of machine-learning models and/or neural networks, efficiently using pipelining, parallel processing, or the like; such a hardware unit may be optimized for such processes by, for instance, including dedicated circuitry for matrix and/or signal processing operations that includes, e.g., multiple arithmetic and/or logical circuit units such as multipliers and/or adders that can act simultaneously and/or in parallel or the like. Such dedicated hardware units 836 may include, without limitation, graphical processing units (GPUs), dedicated signal processing modules, FPGA or other reconfigurable hardware that has been configured to instantiate parallel processing units for one or more specific tasks, or the like, A computing device, processor, apparatus, or module may be configured to instruct one or more dedicated hardware units 836 to perform one or more operations described herein, such as evaluation of model and/or algorithm outputs, one-time or iterative updates to parameters, coefficients, weights, and/or biases, and/or any other operations such as vector and/or matrix operations as described in this disclosure.

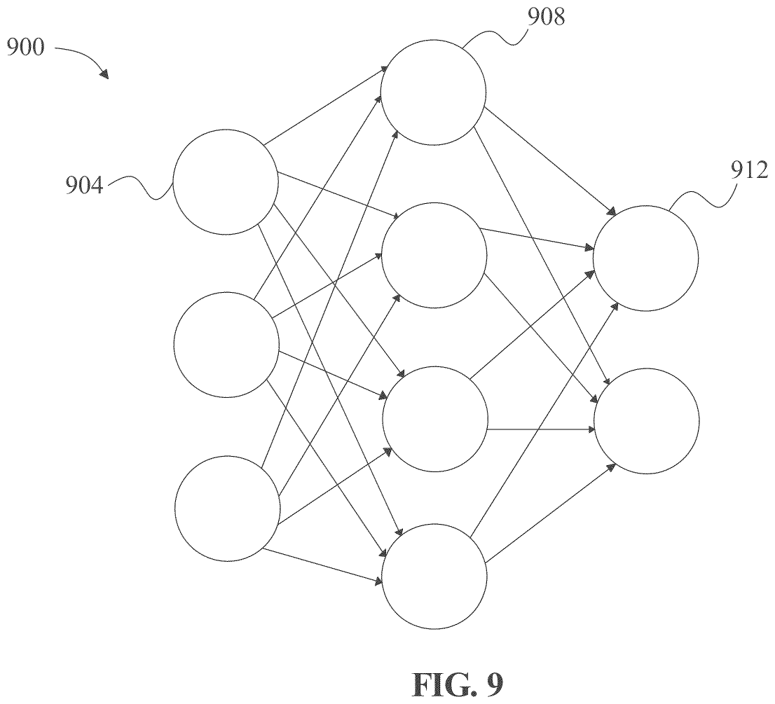

Referring now to FIG. 9, an exemplary embodiment of neural network 900 is illustrated. In some cases, generative model 112 may include a neural network 900. Neural network 900 also known as an artificial neural network, is a network of “nodes,” or data structures having one or more inputs, one or more outputs, and a function determining outputs based on inputs. Such nodes may be organized in a network, such as without limitation a convolutional neural network, including an input layer of nodes 904, one or more intermediate layers 908, and an output layer of nodes 912. Connections between nodes may be created via the process of “training” the network, in which elements from a training dataset are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning. Connections may run solely from input nodes toward output nodes in a “feed-forward” network or may feed outputs of one layer back to inputs of the same or a different layer in a “recurrent network.”

With continued reference to FIG. 9, as a further non-limiting example, a neural network may include a convolutional neural network comprising an input layer of nodes, one or more intermediate layers, and an output layer of nodes. A “convolutional neural network,” for the purpose of this disclosure, is a neural network in which at least one hidden layer is a convolutional layer that convolves inputs to that layer with a subset of inputs known as a “kernel,” along with one or more additional layers such as pooling layers, fully connected layers, and the like. In some cases, CNN may include, without limitation, a deep neural network (DNN) extension. Mathematical (or convolution) operations performed in the convolutional layer may include convolution of two or more functions, where the kernel may be applied to input data through a sliding window approach. In some cases, convolution operations may enable processor 104 to detect local/global patterns, edges, and any other features described herein within biomedical signal such as ECG data. Detected patterns and/or features may be passed through one or more activation functions, such as without limitation, Rectified Linear Unit (ReLU), to introduce non-linearities into the processing step of, for example, and without limitation, identifying biomedical features as described above. Additionally, or alternatively, CNN may also include one or more pooling layers, wherein each pooling layer is configured to reduce the dimensionality of input data while preserving essential features within the input data. In a non-limiting example, CNN may include one or more pooling layer configured to reduce the dimensions of feature maps by applying downsampling, such as max-pooling or average pooling, to small, non-overlapping regions of one or more features. CNN may further include one or more fully connected layers configured to combine features extracted by the convolutional and pooling layers as described above. In some cases, one or more fully connected layers may allow for higher-level pattern recognition. In a non-limiting example, one or more fully connected layers may connect every neuron (i.e., node) in its input to every neuron in its output, functioning as a traditional feedforward neural network layer. In some cases, one or more fully connected layers may be used at the end of CNN to perform high-level reasoning and produce the final output such as, without limitation, at least one biomedical feature. Additionally, or alternatively, each fully connected layer may be followed by one or more dropout layers configured to prevent overfitting, and one or more normalization layers to stabilize the learning process described herein. Training the generative model 128 including a CNN may include selecting a suitable loss function to guide the training process. In a non-limiting example, a loss function that measures the difference between the predicted output and the ground truth may be used, such as, without limitation, mean squared error (MSE) or a custom loss function may be designed for one or more embodiments described herein. Additionally, or alternatively, optimization algorithms, such as stochastic gradient descent (SGD), may then be used to adjust the generative model's 112 parameters to minimize such loss.

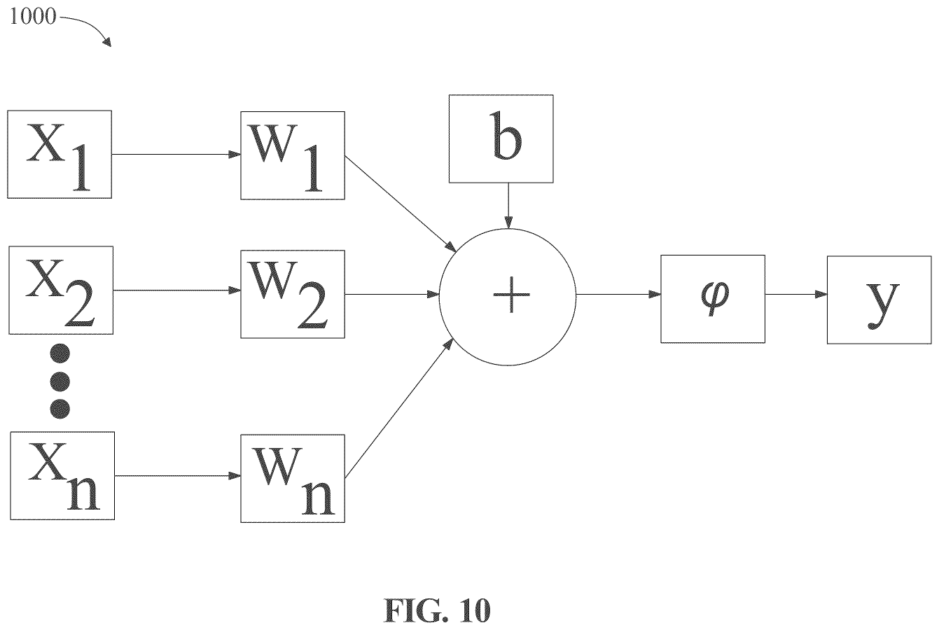

Referring now to FIG. 10, an exemplary embodiment of a node 1000 of a neural network is illustrated. A node may include, without limitation, a plurality of inputs xᵢ that may receive numerical values from inputs to a neural network containing the node and/or from other nodes. Node may perform one or more activation functions to produce its output given one or more inputs, such as without limitation computing a binary step function comparing an input to a threshold value and outputting either a logic 1 or logic 0 output or something equivalent, a linear activation function whereby an output is directly proportional to the input, and/or a non-linear activation function, wherein the output is not proportional to the input. Non-linear activation functions may include, without limitation, a sigmoid function of the form

$$f ( x ) = \frac{1}{1 - {e}^{- x}}$$

given input x, a tanh (hyperbolic tangent) function, of the form

$$\frac{{e}^{x} - {e}^{- x}}{{e}^{x} + {e}^{- x}} ,$$

a tanh derivative function such as ƒ(x)=tanh²(x), a rectified linear unit function such as ƒ(x)=max(0, x), a “leaky” and/or “parametric” rectified linear unit function such as ƒ(x)=max(ax, x) for some a, an exponential linear units function such as

$$f ( x ) = \{ \begin{matrix}x & for x \geq 0 \\ α ( {e}^{x} - 1 ) & for x < 0\end{matrix}$$

for some value of a (this function may be replaced and/or weighted by its own derivative in some embodiments), a softmax function such as

$$f ( {x}_{i} ) = \frac{{e}^{x}}{{\sum }_{i} {x}_{i}}$$

where the inputs to an instant layer are xᵢ, a swish function such as ƒ(x)=x*sigmoid(x), a Gaussian error linear unit function such as f(x)=a(1+tanh(\sqrt{2/π}(x+bx^{r}))) for some values of a, b, and r, and/or a scaled exponential linear unit function such as

$$f ( x ) = λ \{ \begin{matrix}α ( {e}^{x} - 1 ) & for x < 0 \\ x & for x \geq 0\end{matrix} .$$

Fundamentally, there is no limit to the nature of functions of inputs xᵢ that may be used as activation functions. As a non-limiting and illustrative example, node may perform a weighted sum of inputs using weights wᵢ that are multiplied by respective inputs xᵢ. Additionally, or alternatively, a bias b may be added to the weighted sum of the inputs such that an offset is added to each unit in the neural network layer that is independent of the input to the layer. The weighted sum may then be input into a function p, which may generate one or more outputs y. Weight wᵢ applied to an input xᵢ may indicate whether the input is “excitatory,” indicating that it has strong influence on the one or more outputs y, for instance by the corresponding weight having a large numerical value, and/or a “inhibitory,” indicating it has a weak effect influence on the one more inputs y, for instance by the corresponding weight having a small numerical value. The values of weights wᵢ may be determined by training a neural network using training data, which may be performed using any suitable process as described above.

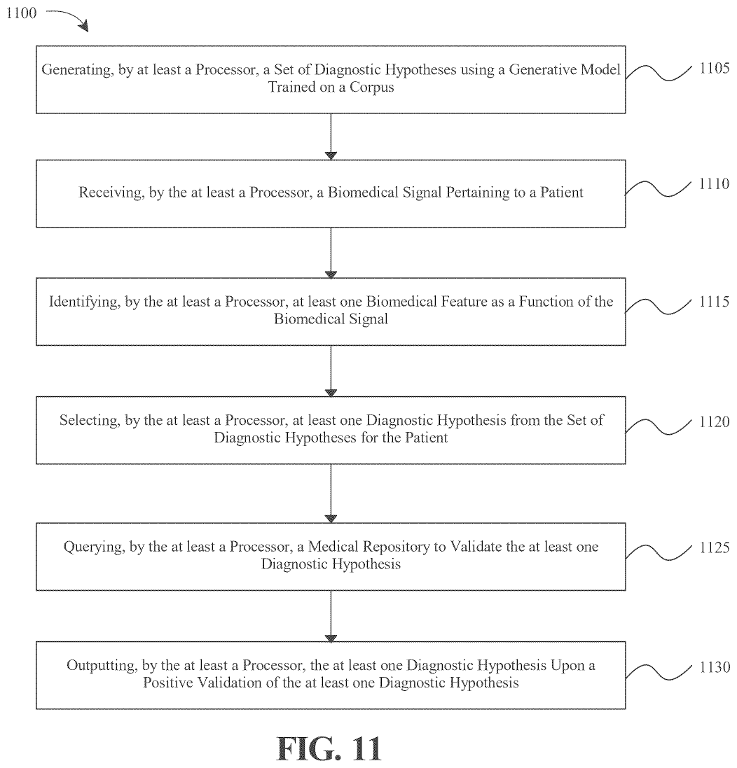

Now referring to FIG. 11, a flow diagram of an exemplary embodiment of a method 1100 for generating diagnostic hypotheses based on ECG data is illustrated. Method 1100 includes a step 1105 of generating, by at least a processor, a set of diagnostic hypotheses using a generative model trained on a corpus, wherein generating the set of diagnostic hypotheses includes creating a plurality of labels, wherein each label of the plurality label represents at least one diagnostic feature associated with one or more diagnostic hypotheses within the set of diagnostic hypotheses. In some embodiments, corpus may include a set of medical literatures. In some embodiments, generating the set of diagnostic hypotheses may include training a large language model (LLM) using the set of medical literatures and generating the set of diagnostic hypotheses using the LLM. Training the LLM may include pre-training the LLM on a general set of medical literatures and fine-tuning the LLM on a special set of medical literatures, wherein the general set of medical literatures and the special set of medical literatures are subsets of the set of medical literatures. This may be implemented, without limitation, as described above with reference to FIGS. 1-10.

With continued reference to FIG. 11, method 1100 includes a step 1110 of receiving, by the at least a processor, a biomedical signal pertaining to a patient. In some embodiments, the biomedical signal may include ECG data. This may be implemented, without limitation, as described above with reference to FIGS. 1-10.

With continued reference to FIG. 11, method 1100 includes a step 1115 of identifying, by the at least a processor, at least one biomedical feature as a function of the biomedical signal. In some embodiments, the at least one biomedical feature may include at least one ECG feature identified from the ECG data. In some embodiments, the at least one ECG feature may include at least one data element describing a cardiac abnormality. In some embodiments, identifying the at least one biomedical feature may include extracting a plurality of biomedical features from the biomedical signal, ranking the plurality of biomedical features based on a set of pre-determined criteria, and identifying the at least one biomedical feature from the plurality of biomedical features based on the rank of the plurality of biomedical features. This may be implemented, without limitation, as described above with reference to FIGS. 1-10.

With continued reference to FIG. 11, method 1100 includes a step 1120 of selecting, by the at least a processor, at least one diagnostic hypothesis from the set of diagnostic hypotheses for the patient by matching the at least one biomedical feature against the at least one diagnostic feature. In some embodiments, selecting the at least one diagnostic hypothesis may include determining, for each diagnostic hypothesis within the set of diagnostic hypotheses, a confidence level as a function of the at least one biomedical feature and selecting the at least one diagnostic hypothesis from the set of diagnostic hypotheses as a function the confidence levels. This may be implemented, without limitation, as described above with reference to FIGS. 1-10.

With continued reference to FIG. 11, method 1100 includes a step 1125 of querying, by the at least a processor, a medical repository in communication with the processor as a function of at least a matched label to validate the at least one diagnostic hypothesis, wherein the medical repository comprises a plurality of electronic health records (EHRs) associated with a plurality of patients. This may be implemented, without limitation, as described above with reference to FIGS. 1-10.

With continued reference to FIG. 11, method 1100 includes a step 1130 of outputting, by the at least a processor, the at least one diagnostic hypothesis upon a positive validation of the at least one diagnostic hypothesis. In some embodiments, validating the at least one diagnostic hypothesis may include retrieving, from the medical repository, one or more EHRs of the plurality of EHRs as a function of the at least a matched label and comparing the at least one diagnostic hypothesis with one or more reference biomedical signals encapsulated in the one or more EHRs, and outputting the at least one diagnostic hypothesis may include outputting the at least one diagnostic hypothesis as a function of the comparison. In some cases, outputting the at least one diagnostic hypothesis may include retrieving one or more medical literatures in relation to the at least one matched label, generating, at the generative model, a diagnostic response as a function of the one or more medical literatures, and displaying, through a user interface at a display device, the diagnostic response. This may be implemented, without limitation, as described above with reference to FIGS. 1-10.

It is to be noted that any one or more of the aspects and embodiments described herein may be conveniently implemented using one or more machines (e.g., one or more computing devices that are utilized as a user computing device for an electronic document, one or more server devices, such as a document server, etc.) programmed according to the teachings of the present specification, as will be apparent to those of ordinary skill in the computer art. Appropriate software coding can readily be prepared by skilled programmers based on the teachings of the present disclosure, as will be apparent to those of ordinary skill in the software art. Aspects and implementations discussed above employing software and/or software modules may also include appropriate hardware for assisting in the implementation of the machine executable instructions of the software and/or software module.

Such software may be a computer program product that employs a machine-readable storage medium. A machine-readable storage medium may be any medium that is capable of storing and/or encoding a sequence of instructions for execution by a machine (e.g., a computing device) and that causes the machine to perform any one of the methodologies and/or embodiments described herein. Examples of a machine-readable storage medium include, but are not limited to, a magnetic disk, an optical disc (e.g., CD, CD-R, DVD, DVD-R, etc.), a magneto-optical disk, a read-only memory “ROM” device, a random access memory “RAM” device, a magnetic card, an optical card, a solid-state memory device, an EPROM, an EEPROM, and any combinations thereof. A machine-readable medium, as used herein, is intended to include a single medium as well as a collection of physically separate media, such as, for example, a collection of compact discs or one or more hard disk drives in combination with a computer memory. As used herein, a machine-readable storage medium does not include transitory forms of signal transmission.

Such software may also include information (e.g., data) carried as a data signal on a data carrier, such as a carrier wave. For example, machine-executable information may be included as a data-carrying signal embodied in a data carrier in which the signal encodes a sequence of instruction, or portion thereof, for execution by a machine (e.g., a computing device) and any related information (e.g., data structures and data) that causes the machine to perform any one of the methodologies and/or embodiments described herein.

Examples of a computing device include, but are not limited to, an electronic book reading device, a computer workstation, a terminal computer, a server computer, a handheld device (e.g., a tablet computer, a smartphone, etc.), a web appliance, a network router, a network switch, a network bridge, any machine capable of executing a sequence of instructions that specify an action to be taken by that machine, and any combinations thereof. In one example, a computing device may include and/or be included in a kiosk.

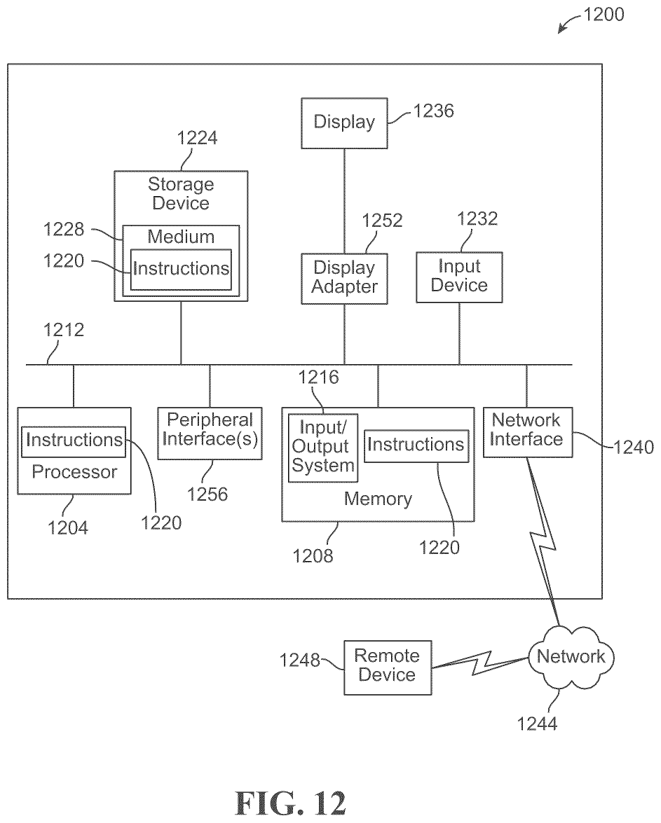

FIG. 12 shows a diagrammatic representation of one embodiment of a computing device in the exemplary form of a computer system 1200 within which a set of instructions for causing a control system to perform any one or more of the aspects and/or methodologies of the present disclosure may be executed. It is also contemplated that multiple computing devices may be utilized to implement a specially configured set of instructions for causing one or more of the devices to perform any one or more of the aspects and/or methodologies of the present disclosure. Computer system 1200 includes a processor 1204 and a memory 1208 that communicate with each other, and with other components, via a bus 1212. Bus 1212 may include any of several types of bus structures including, but not limited to, a memory bus, a memory controller, a peripheral bus, a local bus, and any combinations thereof, using any of a variety of bus architectures.

Processor 1204 may include any suitable processor, such as without limitation a processor incorporating logical circuitry for performing arithmetic and logical operations, such as an arithmetic and logic unit (ALU), which may be regulated with a state machine and directed by operational inputs from memory and/or sensors; processor 1204 may be organized according to Von Neumann and/or Harvard architecture as a non-limiting example. Processor 1204 may include, incorporate, and/or be incorporated in, without limitation, a microcontroller, microprocessor, digital signal processor (DSP), Field Programmable Gate Array (FPGA), Complex Programmable Logic Device (CPLD), Graphical Processing Unit (GPU), general purpose GPU, Tensor Processing Unit (TPU), analog or mixed signal processor, Trusted Platform Module (TPM), a floating point unit (FPU), system on module (SOM), and/or system on a chip (SoC).

Memory 1208 may include various components (e.g., machine-readable media) including, but not limited to, a random-access memory component, a read only component, and any combinations thereof. In one example, a basic input/output system 1216 (BIOS), including basic routines that help to transfer information between elements within computer system 1200, such as during start-up, may be stored in memory 1208. Memory 1208 may also include (e.g., stored on one or more machine-readable media) instructions (e.g., software) 1220 embodying any one or more of the aspects and/or methodologies of the present disclosure. In another example, memory 1208 may further include any number of program modules including, but not limited to, an operating system, one or more application programs, other program modules, program data, and any combinations thereof.

Computer system 1200 may also include a storage device 1224. Examples of a storage device (e.g., storage device 1224) include, but are not limited to, a hard disk drive, a magnetic disk drive, an optical disc drive in combination with an optical medium, a solid-state memory device, and any combinations thereof. Storage device 1224 may be connected to bus 1212 by an appropriate interface (not shown). Example interfaces include, but are not limited to, SCSI, advanced technology attachment (ATA), serial ATA, universal serial bus (USB), IEEE 1394 (FIREWIRE), and any combinations thereof. In one example, storage device 1224 (or one or more components thereof) may be removably interfaced with computer system 1200 (e.g., via an external port connector (not shown)). Particularly, storage device 1224 and an associated machine-readable medium 1228 may provide nonvolatile and/or volatile storage of machine-readable instructions, data structures, program modules, and/or other data for computer system 1200. In one example, software 1220 may reside, completely or partially, within machine-readable medium 1228. In another example, software 1220 may reside, completely or partially, within processor 1204.

Computer system 1200 may also include an input device 1232. In one example, a user of computer system 1200 may enter commands and/or other information into computer system 1200 via input device 1232. Examples of an input device 1232 include, but are not limited to, an alpha-numeric input device (e.g., a keyboard), a pointing device, a joystick, a gamepad, an audio input device (e.g., a microphone, a voice response system, etc.), a cursor control device (e.g., a mouse), a touchpad, an optical scanner, a video capture device (e.g., a still camera, a video camera), a touchscreen, and any combinations thereof. Input device 1232 may be interfaced to bus 1212 via any of a variety of interfaces (not shown) including, but not limited to, a serial interface, a parallel interface, a game port, a USB interface, a FIREWIRE interface, a direct interface to bus 1212, and any combinations thereof. Input device 1232 may include a touch screen interface that may be a part of or separate from display 1236, discussed further below. Input device 1232 may be utilized as a user selection device for selecting one or more graphical representations in a graphical interface as described above.

A user may also input commands and/or other information to computer system 1200 via storage device 1224 (e.g., a removable disk drive, a flash drive, etc.) and/or network interface device 1240. A network interface device, such as network interface device 1240, may be utilized for connecting computer system 1200 to one or more of a variety of networks, such as network 1244, and one or more remote devices 1248 connected thereto. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network, such as network 1244, may employ a wired and/or a wireless mode of communication. In general, any network topology may be used. Information (e.g., data, software 1220, etc.) may be communicated to and/or from computer system 1200 via network interface device 1240.

Computer system 1200 may further include a video display adapter 1252 for communicating a displayable image to a display device, such as display device 1236. Examples of a display device include, but are not limited to, a liquid crystal display (LCD), a cathode ray tube (CRT), a plasma display, a light emitting diode (LED) display, and any combinations thereof. Display adapter 1252 and display device 1236 may be utilized in combination with processor 1204 to provide graphical representations of aspects of the present disclosure. In addition to a display device, computer system 1200 may include one or more other peripheral output devices including, but not limited to, an audio speaker, a printer, and any combinations thereof. Such peripheral output devices may be connected to bus 1212 via a peripheral interface 1256. Examples of a peripheral interface include, but are not limited to, a serial port, a USB connection, a FIREWIRE connection, a parallel connection, and any combinations thereof.

The foregoing has been a detailed description of illustrative embodiments of the invention. Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present invention. Additionally, although particular methods herein may be illustrated and/or described as being performed in a specific order, the ordering is highly variable within ordinary skill to achieve methods, systems, and software according to the present disclosure. Accordingly, this description is meant to be taken only by way of example, and not to otherwise limit the scope of this invention.

Exemplary embodiments have been disclosed above and illustrated in the accompanying drawings. It will be understood by those skilled in the art that various changes, omissions, and additions may be made to that which is specifically disclosed herein without departing from the spirit and scope of the present invention.

1. An apparatus for generating diagnostic hypotheses based on biomedical signal data, the apparatus comprising:
at least a processor; and
a memory communicatively connected to the at least a processor, wherein the memory contains instructions configuring the at least a processor to:
generate, using a large language model (LLM) trained on a corpus comprising a set of medical literatures, a set of diagnostic hypotheses, wherein each diagnostic hypothesis of the set of diagnostic hypotheses comprises one or more of at least an inclusion criterion and at least an exclusion criterion, wherein generating the set of diagnostic hypotheses comprises:
creating a plurality of labels, wherein each label of the plurality of labels represents at least one diagnostic feature associated with one or more diagnostic hypotheses within the set of diagnostic hypotheses; and
outputting the set of diagnostic hypotheses using the LLM;
receive a biomedical signal comprising electrocardiogram (ECG) data pertaining to a patient;
identify at least one biomedical feature comprising at least one ECG feature as a function of the ECG data, wherein identifying the at least one ECG feature comprises:
inputting the ECG data into at least a generative model; and
outputting the at least one ECG feature as a function of the at least a generative model and the ECG data;
select at least one diagnostic hypothesis from the set of diagnostic hypotheses for the patient by matching the at least one ECG feature against the at least one diagnostic feature;
query, as a function of the at least one ECG feature matched against the at least one diagnostic feature, a medical repository, in communication with the processor, to validate the at least one matched diagnostic hypothesis, wherein the medical repository comprises a plurality of electronic health records (EHRs) comprising a plurality of electrocardiograms (ECGs) associated with a plurality of patients;
receive, as a function of the query, query results from the medical repository comprising the plurality of EHRs; and
output the at least one diagnostic hypothesis and the query results.

2. (canceled)

3. (canceled)

4. (canceled)

5. (canceled)

6. The apparatus of claim 1, wherein the diagnostic hypothesis represents a cohort defined by one or more of an inclusion criterion and an exclusion criterion; and
the query results represent a subset of EHRs belonging to the cohort.

7. The apparatus of claim 1, wherein identifying the at least one biomedical feature comprises:
extracting a plurality of biomedical features from the biomedical signal;
ranking the plurality of biomedical features based on a set of pre-determined criteria; and
identifying the at least one biomedical feature from the plurality of biomedical features based on the rank of the plurality of biomedical features.

8. The apparatus of claim 1, wherein selecting the at least one diagnostic hypothesis comprises:
determining, for each diagnostic hypothesis within the set of diagnostic hypotheses, a confidence level as a function of the at least one biomedical feature; and
selecting the at least one diagnostic hypothesis from the set of diagnostic hypotheses as a function of the confidence levels.

9. The apparatus of claim 1, wherein:
validating the at least one diagnostic hypothesis comprises:
retrieving, from the medical repository, one or more EHRs of the plurality of EHRs as a function of the at least a matched label; and
comparing the at least one diagnostic hypothesis with one or more reference biomedical signals encapsulated in the one or more EHRs; and
outputting the at least one diagnostic hypothesis comprises outputting the at least one diagnostic hypothesis as a function of the comparison.

10. The apparatus of claim 1, wherein outputting the at least one diagnostic hypothesis comprises:
retrieving one or more medical literatures in relation to the at least a matched label;
generating, at the LLM, a diagnostic response as a function of the one or more medical literatures; and
displaying, through a user interface at a display device, the diagnostic response.

11. A method for generating diagnostic hypotheses based on electrocardiogram (ECG) data, the method comprising:
generating, by at least a processor, a set of diagnostic hypotheses using a large language model (LLM) trained on a corpus comprising a set of medical literatures, wherein generating the set of diagnostic hypotheses using the large language model (LLM) comprises:
creating a plurality of labels, wherein each label of the plurality of labels represents at least one diagnostic feature associated with one or more diagnostic hypotheses within the set of diagnostic hypotheses, wherein each diagnostic hypothesis of the set of diagnostic hypotheses comprises one or more of at least an inclusion criterion and at least an exclusion criterion; and
outputting the set of diagnostic hypotheses using the LLM;
receiving, by the at least a processor, a biomedical signal comprising electrocardiogram (ECG) data pertaining to a patient;
identifying, by the at least a processor, at least one biomedical feature comprising at least one ECG feature as a function of the ECG data, wherein identifying the at least one ECG feature comprises:
inputting the ECG data into at least a generative model; and
outputting the at least one ECG feature as a function of the at least a generative model and the ECG data;
selecting, by the at least a processor, at least one diagnostic hypothesis from the set of diagnostic hypotheses for the patient by matching the at least one ECG feature against the at least one diagnostic feature;
querying, by the at least a processor, as a function of the at least one ECG feature matched against the at least one diagnostic feature, a medical repository in communication with the processor as a function of at least a matched label to validate the at least one diagnostic hypothesis, wherein the medical repository comprises a plurality of electronic health records (EHRs) associated with a plurality of patients; and
outputting, by the at least a processor, the at least one diagnostic hypothesis upon a positive validation of the at least one diagnostic hypothesis.

12. (canceled)

13. (canceled)

14. (canceled)

15. (canceled)

16. The method of claim 11, wherein the diagnostic hypothesis represents a cohort defined by one or more of an inclusion criterion and an exclusion criterion; and
the query results represent a subset of EHRs belonging to the cohort.

17. The method of claim 11, wherein identifying the at least one biomedical feature comprises:
extracting a plurality of biomedical features from the biomedical signal;
ranking the plurality of biomedical features based on a set of pre-determined criteria; and
identifying the at least one biomedical feature from the plurality of biomedical features based on the rank of the plurality of biomedical features.

18. The method of claim 11, wherein selecting the at least one diagnostic hypothesis comprises:
determining, for each diagnostic hypothesis within the set of diagnostic hypotheses, a confidence level as a function of the at least one biomedical feature; and
selecting the at least one diagnostic hypothesis from the set of diagnostic hypotheses as a function of the confidence levels.

19. The method of claim 11, wherein:
validating the at least one diagnostic hypothesis comprises:
retrieving, from the medical repository, one or more EHRs of the plurality of EHRs as a function of the at least a matched label; and
comparing the at least one diagnostic hypothesis with one or more reference biomedical signals encapsulated in the one or more EHRs; and
outputting the at least one diagnostic hypothesis comprises outputting the at least one diagnostic hypothesis as a function of the comparison.

20. The method of claim 11, wherein outputting the at least one diagnostic hypothesis comprises:
retrieving one or more medical literatures in relation to the at least a matched label;
generating, at the LLM, a diagnostic response as a function of the one or more medical literatures; and
displaying, through a user interface at a display device, the diagnostic response.

21. The apparatus of claim 1, wherein outputting the at least a diagnostic hypothesis and the query results is through a user interface wherein the user interface comprises an event-handler-driven interface comprising a cross-session state variable, wherein the at least a processor stores an identifier of a the at least one diagnostic hypothesis in the cross-session state variable as an obfuscated data element within a cookie that further contains an identifier of a requesting entity, and upon a subsequent session, automatically repopulates the user interface with the stored hypothesis to reduce repeated data entry.

22. The method of claim 11, wherein outputting the at least a diagnostic hypothesis and the query results is through a user interface wherein the user interface comprises an event-handler-driven interface comprising a cross-session state variable, wherein the at least a processor stores an identifier of a the at least one diagnostic hypothesis in the cross-session state variable as an obfuscated data element within a cookie that further contains an identifier of a requesting entity, and upon a subsequent session, automatically repopulates the user interface with the stored hypothesis to reduce repeated data entry.
